(19) United States
(12) Patent Application Publication
(10) Pub. No.: US 2002/0176865 A1
(43) Pub. Date: Nov. 28, 2002

(54) Isolated nucleic acid molecules coding for tumor rejection antigen precursors of members of the MAGE-C and MAGE-B families and uses thereof

(76) Inventors: Sophie Lucas, Brussels (BE); Thierry Boon-Falleur, Brussels (BE)

Correspondence Address: FULBRIGHT & JAWORSKI L.L.P., Mary Anne Schofield, Market Square, 801 Pennsylvania Avenue, N.W., Washington, DC, 20004-2615, US

(21) Appl. No.: 10/085,108

(22) Filed: Mar. 1, 2002

Related U.S. Application Data
(62) Division of application No. 09/501,104, filed on Feb. 9, 2000.
Application No. 09/501,104 is a continuation-in-part of application No. 09/468,433, filed on Dec. 17, 1999.
Application No. 09/468,433 is a continuation-in-part of application No. 09/066,281, filed on Apr. 24, 1998.
Application No. 09/066,281 is a continuation-in-part of application No. 08/845,528, filed on Apr. 25, 1997, now Pat. No. 6,027,924.

(51) Int. Cl. A61K 39/395; C07H 21/04; C12P 21/02; C12N 5/06

(52) U.S. Cl. 424/185.1; 530/350; 435/69.3; 435/320.1; 435/325; 536/23.2

(57) ABSTRACT

The invention relates to isolated nucleic acid molecules which code for antigens expressed by tumor cells which maybe recognized by cytotoxic T cells, leading to lysis of the tumor cells which express it. This invention also relates to vectors which are designed to encode the antigen expressed by tumor cells and also to cells transfected by the nucleic acid molecules or vectors which comprise the nucleic acid molecules. Various therapeutic and diagnostic uses arising out of the properties of the nucleic acid molecules and the antigens for which these code are also part of this invention.

### RELATED APPLICATION

This application is a continuation-in-part of co-pending application entitled “Isolated Nucleic Acid Molecules Coding for Tumor Rejection Antigen Precursors of Members of the MAGE-C and MAGE-B Families and Uses Thereof”, inventors Sophia Lucas and Thierry Boon-Falleur filed Dec. 17, 1999 (Express Mail no. EM004582641US) (Ser. No. not yet assigned), which is a continuation in part of application Ser. No. 09/066,281 filed Apr. 24, 1998, which is a continuation-in-part of Ser. No. 08/845,528 filed on Apr. 25, 1997, all of which are incorporated in their entirety by reference.

### FIELD OF THE INVENTION

This invention relates to nucleic acid molecules which code for tumor rejection antigen precursors of the MAGE-C and MAGE-B FAMILIES. More particularly, the invention concerns nucleic acid molecules which encode tumor rejection antigen precursors which can be processed, inter alia, into peptides presented by many MHC molecules, such as HLA-A1 and its alleles, HLA-A2, HLA-Cw*1601, HLA-B44, and so forth. Preferred embodiments are MAGE-C3, MAGE-B5 and MAGE-B6. These nucleic acid molecules are expressed in a variety of tumors and in normal testis cells, but are not expressed by other normal cells.

### BACKGROUND AND PRIOR ART

The process by which the mammalian immune system recognizes and reacts to foreign or alien materials is a complex one. An important facet of the system is the T lymphocyte, or “T cell” response. This response requires that T cells recognize and interact with complexes of cell surface molecules, referred to as human leukocyte antigens (“HLA”), or major histocompatibility complexes (“MHCs”), and peptides. The peptides are derived from larger molecules which are processed by the cells which also present the HLA/MHC molecule. See in this regard Male et al., Advanced Immunology (J.P. Lipincott Company, 1987), especially chapters 6-10. The interaction of T cells and HLA/peptide complexes is restricted, requiring a T cell specific for a particular combination of an HLA molecule and a peptide. If a specific T cell is not present, there is no T cell response even if its partner complex is present. Similarly, there is no response if the specific complex is absent, but the T cell is present. This mechanism is involved in the immune system's response to foreign materials, in autoimmune pathologies, and in responses to cellular abnormalities. Much work has focused on the mechanisms by which proteins are processed into the HLA binding peptides. See in this regard, Barinaga, Science 257:880 (1992); Fremont et al., Science 257:919 (1992); Matsumura et al., Science 257:927 (1992); Latron et al., Science 257:964 (1992).

The mechanism by which T cells recognize cellular abnormalities has also been implicated in cancer. For example, in PCT application PCT/US92/04354, filed May 22, 1992, published on Nov. 26, 1992, and incorporated by reference, a family of genes is disclosed, which are processed into peptides which, in turn, are expressed on cell surfaces, which can lead to lysis of the tumor cells by specific cytolytic T lymphocytes (“CTLs”). The genes are said to code for “tumor rejection antigen precursors” or “TRAP” molecules, and the peptides derived therefrom are referred to as “tumor rejection antigens” or “TRAs.” See Traversari et al., Immunogenetics 35:145 (1992); van der Bruggen et al., Science 254:1643 (1991), for further information on this family of genes. Also, see U.S. Pat. Nos. 5,342,774 and 5,462,871 incorporated by reference in their entirety.

In U.S. Pat. No. 5,405,940 the disclosure of which is incorporated by reference, it is explained that the MAGE-1 gene codes for a tumor rejection antigen precursor, which is processed to nonapeptides which are presented by the HLA-A1 molecule. The nonapeptides which bind to HLA-A1 follow a “rule” for binding in that a motifis satisfied. In this regard, see, e.g., PCT/US93/07421; Falketal., Nature 351:290-296 (1991); Engelhard, Ann Rev. Immunol. 12:181-207 (1994); Ruppertet al., Cell 74:929-937 (1993); Rotzschke et al., Nature 348:252-254 (1990); Bjorkman et al., Nature 329:512-518(1987); Traversari et al., J. Exp. Med. 176:1453-1457 (1992). The reference teaches that given the known specificity of particular peptides for particular HLA molecules, one should expect a particular peptide to bind to one HLA molecule, but not to others. Because different individuals possess different HLA phenotypes, identification of a particular peptide as being a partner for a particular HLA molecule has diagnostic and therapeutic ramifications, only for individuals with that particular HLA phenotype. There is a need for further work in the area, because cellular abnormalities are not restricted to one particular HLA phenotype, and targeted therapy requires some knowledge of the phenotype of the abnormal cells at issue.

In U.S. patent application Ser. No. 288,977, filed Aug. 11, 1994 now U.S. Pat. No. 5,629,166 and incorporated by reference, the fact that the MAGE-1 expression product is processed to a second TRA is disclosed. This second TRA is presented by HLA-Cw*1601 molecules. The disclosure shows that a given TRAP can yield a plurality of TRAs, each of which will satisfy a motif rule for binding to an MHC molecule.

In U.S. patent applications Ser. Nos. 08/658,578 and 08/846,111 filed Jun. 5, 1996 and Apr. 25, 1997 respectively and incorporated herein by reference, members of another MAGE family, i.e., the MAGE-B family are disclosed. These family members are located on the Xp arm of the X chromosome in contrast to the previously identified MAGE-A family members, which had all been found on the Xq arm.

In U.S. patent application Ser. No. 994,928, filed Dec. 22, 1992, and incorporated by reference herein teaches that tyrosinase, a molecule which is produced by some normal cells (e.g., melanocytes), is processed in tumor cells to yield peptides presented by HLA-A2 molecules.

In U.S. patent application Ser. No. 08/032,978, filed Mar. 18, 1993, and incorporated by reference in its entirety, a second TRA, not derived from tyrosinase is taught to be presented by HLA-A2 molecules. The TRA is derived from a TRAP, but is coded for by a non-MAGE gene. This disclosure shows that a particular HLA molecule may present TRAs derived from different sources.

In U.S. patent application Ser. No. 08/079,110, filed Jun. 17, 1993 now U.S. Pat. No. 5,571,711 issued Jan. 5, 1996 and incorporated by reference herein, an unrelated tumor rejection antigen precursor, the so-called “BAGE” precursor is described. The BAGE precursor is not related to the MAGE family. In U.S. patent applications Ser. No. 08/096,039 and Ser. No. 08/250,162, both of which are incorporated by reference, a non-MAGE TRAP precursor, GAGE, is also disclosed.

U.S. application Ser. No. 08/316,231 filed Sep. 30, 1994, discloses additional tumor rejection antigen precursors. These tumor rejection antigen precursors are referred to as “DAGE” tumor rejection antigen precursors. They do not show homology to the MAGE, the BAGE, or GAGE family of genes.

The work which is presented by the papers, patent, and patent applications cited supra deals, in large part, with the MAGE, BAGE, GAGE, and DAGE family of genes. The present invention relates to nucleic acid molecules of the MAGE-C and MAGE-B families encoding MAGE-related tumor rejection antigen precursors, i.e., MAGE-C1 MAGE-C2, MAGE-C3, MAGE-B5 and MAGE-B6, and to the tumor rejection antigen precursors and tumor rejection antigens themselves. The invention also relates to applications of both nucleic acid and protein molecules.

The invention is elaborated upon further in the disclosure which follows.

### BRIEF DESCRIPTION OF THE FIGURES

FIGS. 1A-F is the nucleotide sequence of MAGE-C1 cDNA. The position of various nucleotide sense and antisense primers are indicated.

FIGS. 2A-F depicts a comparison of the nucleotide sequences of MAGE-C1 and MAGE-A1.

FIG. 3 is a comparison of gene MAGE-C1 with isolated cDNA clone MAGE-C1 and published gene MAGE-A1. Exons appear as boxes and are numbered from I to III (MAGE-A1) or IV (MAGE-C1). Introns appear as lines. Deletion in the cDNA clone as compared to gene MAGE-C1 appears as a blank. Similar regions between genes MAGE-A1 and MAGE-C1 are indicated by shaded areas. Open reading frames are indicated by dark boxes inside the exons. Repeated segments in gene MAGE-C1 are shown as a hatched box. Important restriction sites are indicated (B: BamHI, D: Dpnll, E: EcoRl, P: Pstl, X: Xbal), as well as positions of two pairs of oligonucleotides (SL33/SL34, and SL38/SL43). Asterix upstream from MAGE-C1 exon I show localization of the Sp1 and the 2 Ets consensus recognition sequences. The position of the XbaI-EcoRl cDNA probe is also indicated.

FIG. 4 is a schematic representation of genes MAGE-C1, MAGE-C2, and MAGE-A1. Exons appear as open boxes, introns as lines. Open reading frames are represented by dark boxes. Regions of homology between MAGE-C2 and the two other genes are represented by shaded areas. Important PCR primers are indicated by arrows. The hatched box represents the repetitive region found in gene MAGE-C1.

FIG. 5 is the nucleotide sequence of MAGE-C3, the sequence corresponds to nucleotides 3761 to 4801 of PAC clone 232G24 (GenBank accession number AL022152). The sequence has been renumbered in this figure, taking as nucleotide number 1 the first nucleotide of the start codon. Primers SL 164 and SL 165 that were used in the RT-PCR assay are indicated.

FIG. 6 is the amino acid sequence of MAGE-C3.

FIG. 7 is the nucleotide sequence of MAGE-B5, this sequence is the antiparallel sequence corresponding to nucleotides 123358 to 124185 of BAC clone 46604 (GenBank accession number AC005297). The sequence has been renumbered in this figure, taking the first nucleotide of the start codon as nucleotide number 1. Primers SL189 and SL190 that were used in the RT-PCR assay are indicated.

FIG. 8 is the amino acid sequence of MAGE-B5.

FIG. 9 is the nucleotide sequence of MAGE-B6, this sequence is the antiparallel sequence corresponding to nucleotides 146417 to 147640 of BAC clone 46604 (GenBank accession number AC005297). The sequence has been renumbered in this figure, taking the first nucleotide of the start codon as nucleotide number 1. Primers SL191 and SL192 that were used in the RT-PCR assay are indicated.

FIG. 10 is the amino acid sequence of MAGE-B6.

### DETAILED DESCRIPTION

Many human tumor antigens identified so far are encoded by genes, such as MAGE, BAGE, and GAGE, which share a common expression pattern: they are expressed in testis (and sometimes placenta), but in no other normal tissue, and are reactivated in various tumor types. This type of antigen is of particular interest for tumor immunotherapy.

As an alternative to the identification of tumor antigens by cloning genes coding for antigens known to be recognized by antitumor cytolytic T lymphocytes (CTL), we searched directly for new genes expressed specifically in tumors, as such genes could provide a source of tumor-specific antigens. Using a PCR based subtractive hybridization technique called Representational Difference Analysis applied to cDNA (Hubank and Shatz, “Identifying Differences in mRNA Expression By Representational Difference Analysis of cDNA,” Nucleic Acids Res. 22:5640-5648 (1994)), we identified new members of the MAGE gene family, which we describe herein.

We also performed a search of nucleotide databanks for MAGE-related sequences. Search for nucleotide sequences encoding proteins homologous to protein MAGE-A10 was performed with the tblastn program on the Internet server of the National Center for Biotechnology Information (http://www.ncbi.nlm.nih.gov). This search led to the identification of three new MAGE-type genes that encode new MAGE-type proteins.

A first new MAGE-type gene was identified in genomic nucleotide sequence data that was produced by the Human Chromosome X Sequencing Group at the Sanger Center and can be obtained from http://www.ncbi.nlm.nih.gov/Entrez/nucleotide.html/ under accession number AL022152. This sequence is from clone 232G24 of the P1 artificial chromosome library RPCI6 constructed at the Roswell Park Cancer Institute by the group of Pieter de Jong (for further details see http://bacpac.med.buffalo.edu/). This PAC clone has been mapped to Xq27.1-Xq27.3, a region of the X chromosome that contains also genes MAGE-C1 and MAGE-C2. An open reading frame that encompasses nucleotides 3761 to 4801 of the PAC clone encodes a new protein of 346 amino-acids that shares similarities with all known MAGE proteins. Maximum homology (56% amino-acid identities) is observed with proteins MAGE-C1 and MAGE-C2.

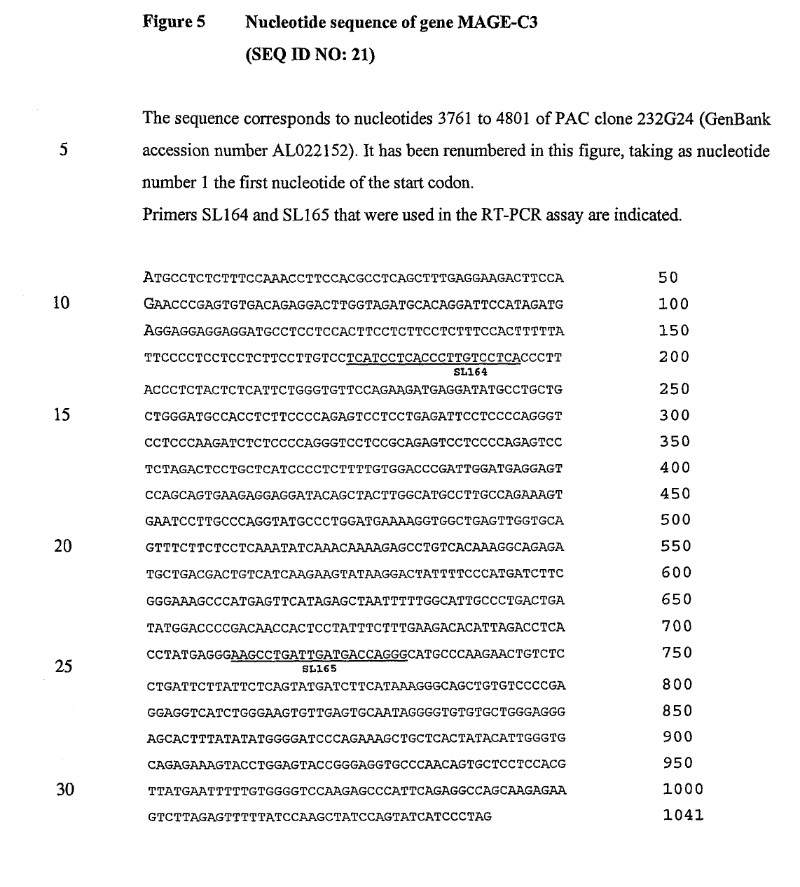

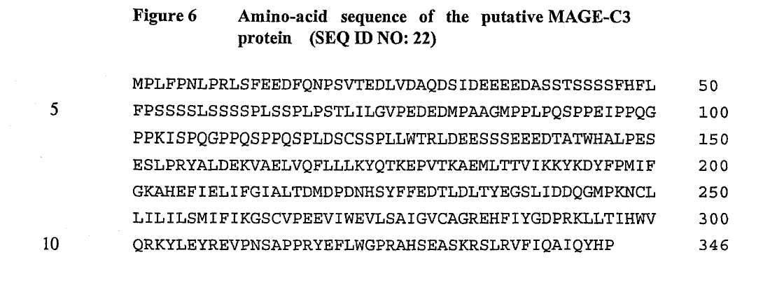

Therefore, this new gene was named MAGE-C3. FIG. 5 shows the nucleotide sequence of gene MAGE-C3. FIG. 6 shows the amino-acid sequence of the encoded MAGE-C3 protein.

Two additional MAGE-type genes were identified in genomic nucleotide sequence data that was produced by the Human Genome Sequencing Center at the Baylor College of Medicine (Houston, Tex., USA) and can be obtained under accession number AC005297. The sequence is from clone 46604 of the Bacterial Artificial Chromosome library RPCI11 constructed at the Roswell Park Cancer Institute by the group of Pieter de Jong. This BAC clone has been mapped to Xp22, a region of the X chromosome located near the region that contains the four known genes of the MAGE-B family. Two open reading frames located in the sequence of the BAC clone encode new proteins that share similarities with other known MAGE proteins. Maximum homology is observed with proteins of the MAGE-B family, and therefore, the two new genes were named MAGE-B5 and MAGE-B6.

The examples of this invention show the isolation of nucleic acid molecules which code for tumor rejection antigen precursors (“TRAP”) MAGE-C1, MAGE-C2, MAGE-C3, MAGE-B5 and MAGE B6. These TRAP encoding molecules share partial homology with the MAGE family coding sequences described in the references set forth supra. Hence, one aspect of this invention is an isolated nucleic acid which encodes a protein comprising the amino acid sequence encoded by the nucleotide sequence set forth in SEQ ID NO: 21 or in SEQ ID NO: 23 or in SEQ ID NO: 25. Preferably, the nucleic acid molecule is a cDNA molecule. SEQ ID NO: 21, SEQ ID NO: 23, and SEQ ID NO: 25 are not previously known MAGE, BAGE, or GAGE coding sequences, as will be seen by comparing it to the sequence of any of these genes as described in the cited references.

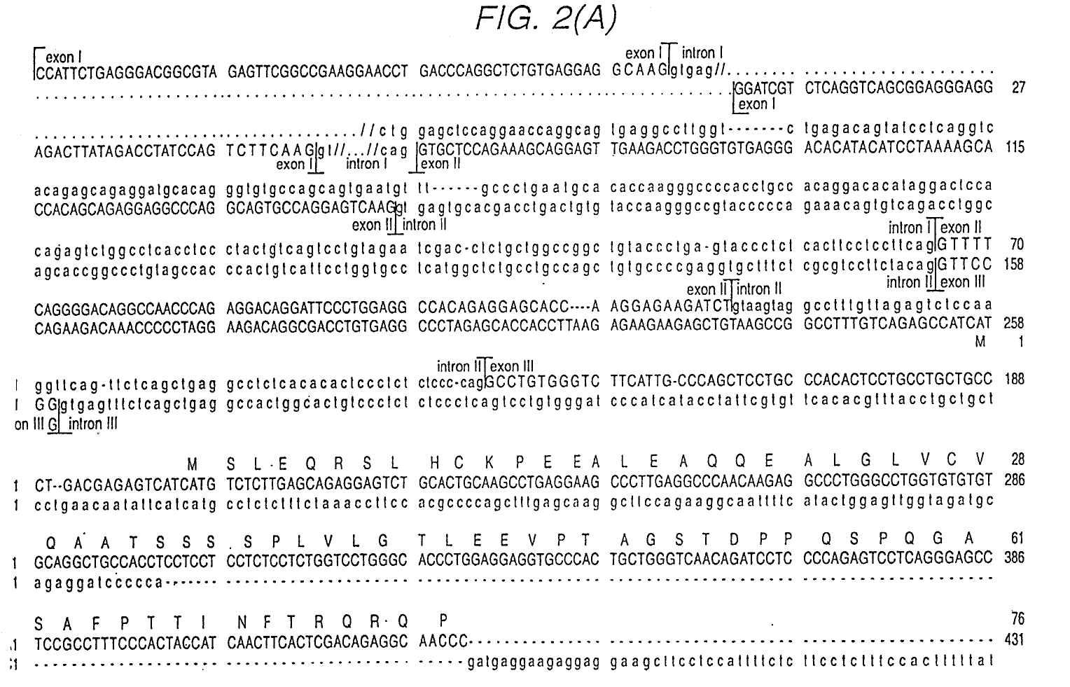

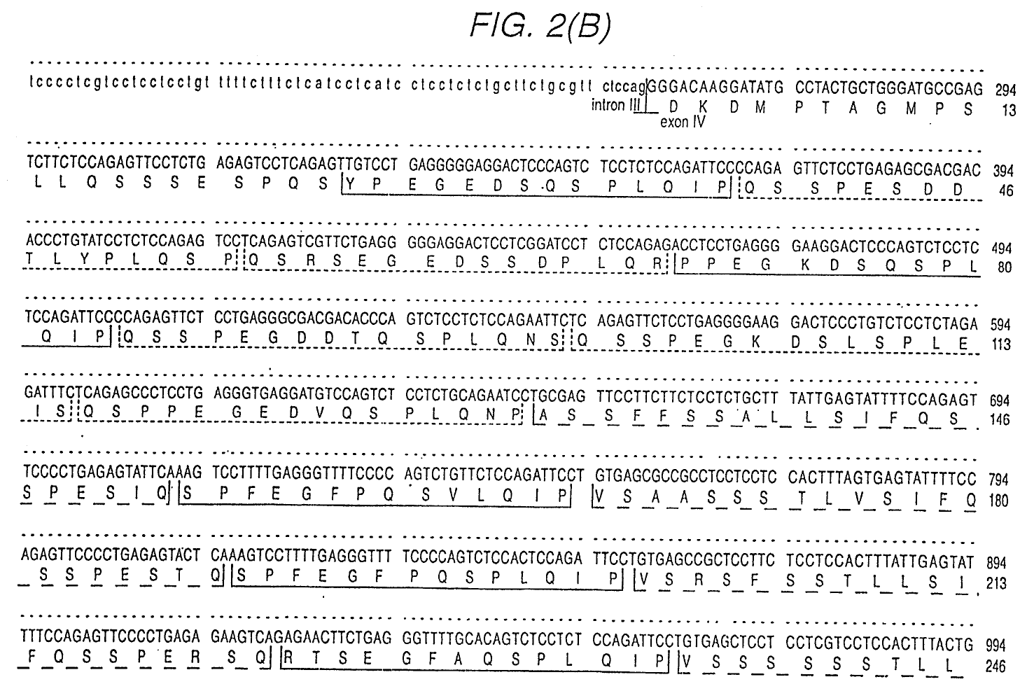

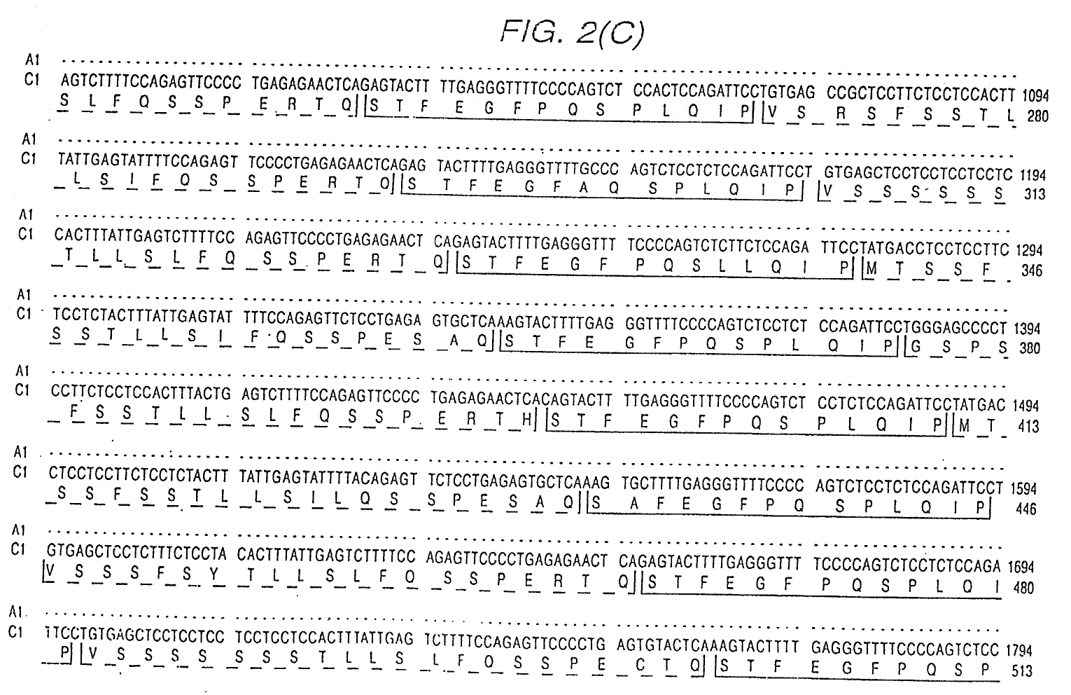

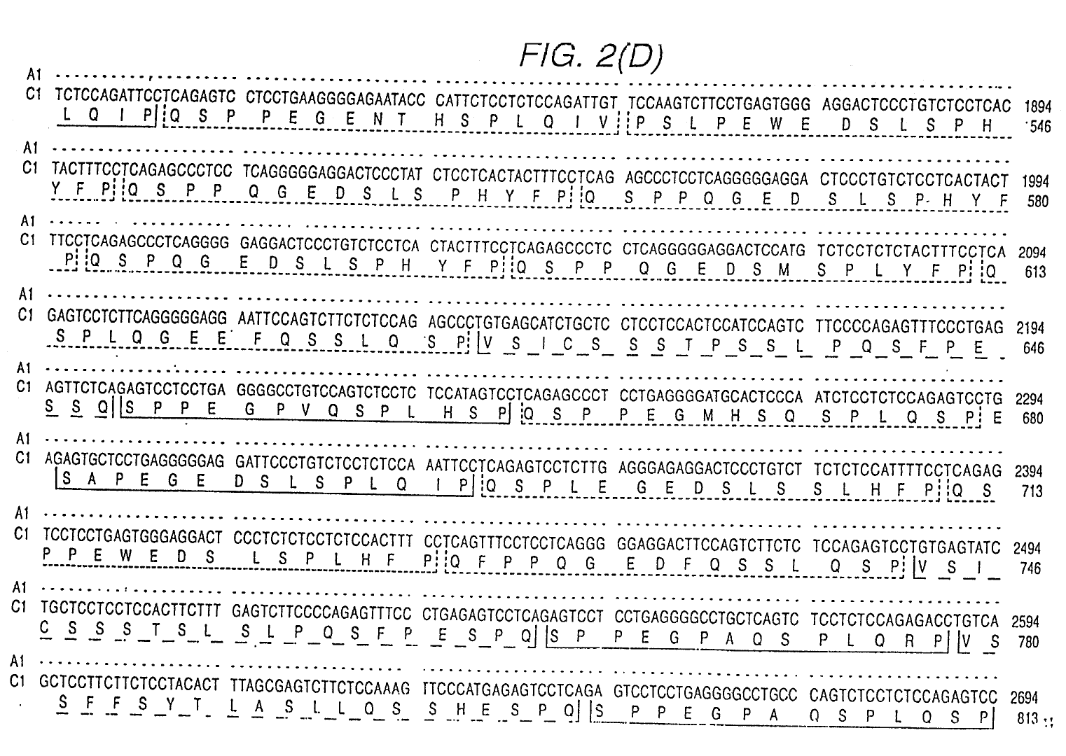

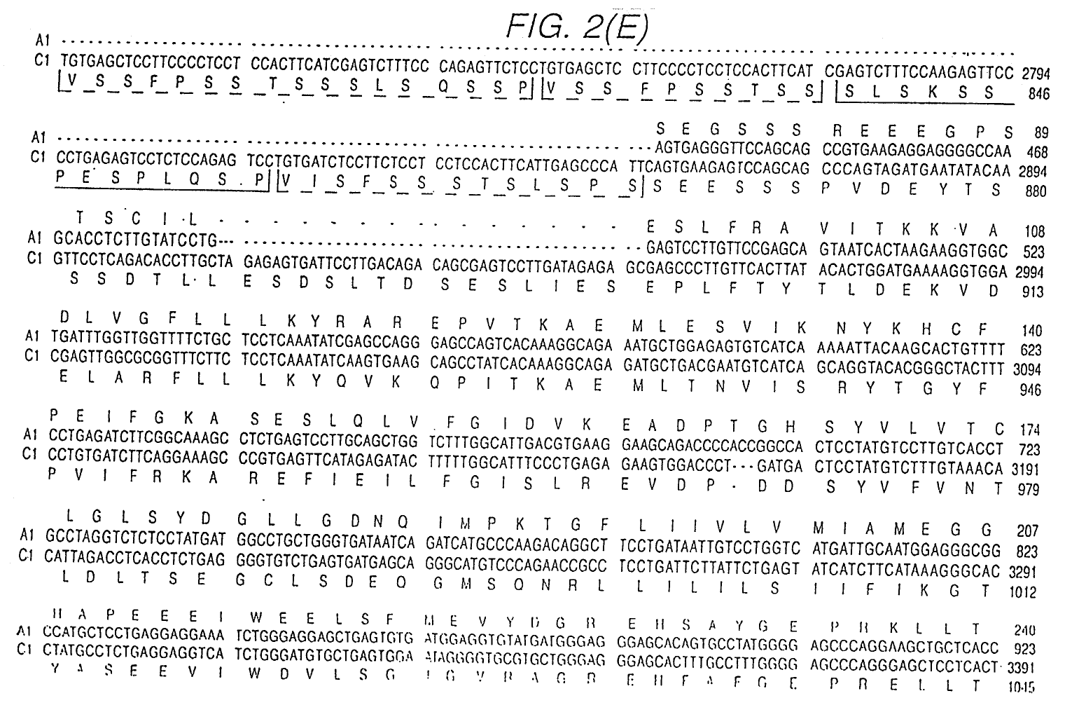

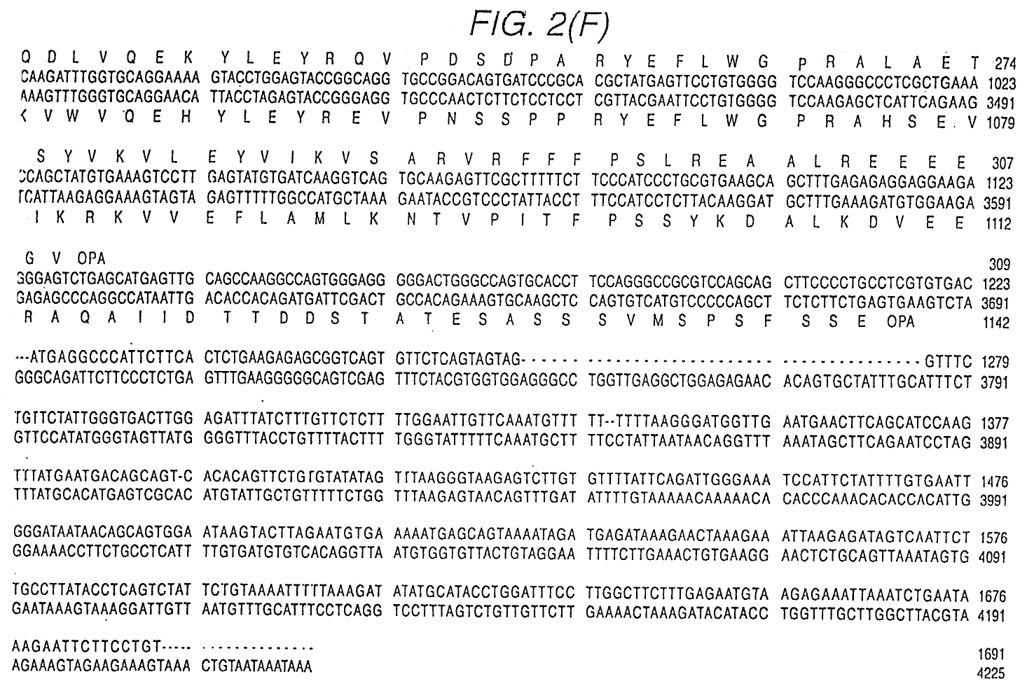

Also, disclosed are those nucleic acid molecules having the nucleotide sequence of nt 1-2815 and nt 2816-4225 of SEQ ID NO: 9 and a nucleic acid molecule, which codes for a tumor rejection antigen precursor and hybridizes to a nucleic acid molecule having the nucleotide sequence 1-2815 of SEQ ID NO: 9 but does not hybridize to nucleic acid molecules having the nucleotide sequence of SEQ ID NO: 8, i.e., the MAGE-A1 nucleotide sequence as set forth in FIG. 2, under stringent conditions. A further embodiment of this invention is a nucleic acid molecule which codes for a tumor rejection antigen precursor and hybridizes to a nucleic acid molecule having the nucleotide sequence 261-2856 of SEQ ID NO: 9 but does not hybridize to nucleic acid molecules having the nucleotide sequence of SEQ ID NO: 8. The term “stringent conditions” as used herein, refers to hybridization in 5× SSC, 0.1% SDS, 5× Denhardt's reagent at 65° C., overnight, followed by two washes at room temperature for 20 minutes, in 2× SSC and 0.1% SDS, and one wash for 20 minutes in 2× SSC and 0.1% SDS at 65° C., and one wash in 0.2× SSC, 0.1% SDS at 65° C. There are other conditions, reagents, and so forth which can be used, which result in the same or higher degree of stringency. The skilled artisan will be familiar with such conditions and, thus, they are not given here.

Another embodiment of this invention is an isolated nucleic acid molecule which codes for a tumor rejection antigen precursor and hybridizes under stringent conditions to a nucleic acid molecule having the nucleotide sequence set forth in SEQ ID NO: 21 (MAGE-C3). A still further aspect of this invention is an isolated nucleic acid molecule which codes for a tumor rejection antigen precursor and hybridizes under stringent conditions to a nucleic acid molecule having the nucleotide sequence set forth in SEQ ID NO: 23 (MAGE-B5). Another aspect of this invention is an isolated nucleic acid molecule which codes for a tumor rejection antigen precursor and hybridizes under stringent conditions to a nucleic acid molecule having the nucleotide sequence set forth in SEQ ID NO: 25 (MAGE-B6).

A further aspect of this invention is an isolated nucleic acid molecule which codes for a tumor rejection antigen precursor and has at least about 79% sequence identity with the cDNA sequence of MAGE-C3 (SEQ ID NO: 21), particularly with nucleotides 472-1023 of SEQ ID NO: 21, with the proviso that the nucleic acid molecule is not MAGE-C1 or MAGE-C2.

The widespread distribution in the expression of MAGE-C1, MAGE-C2, MAGE-C3, MAGE-B5 and MAGE-B6 in tumor cells and not in normal cells, demonstrates that the isolated nucleic acid molecules can be used as diagnostic probes to determine the presence of abnormal, e.g., tumor, cells which express MAGE-C1, MAGE-C2, MAGE-C3, MAGE-B5 or MAGE-B6 related sequences. The identification of seminoma with MAGE-C1 was 100% (Table 2) so on a very basic level, the isolated nucleic acid molecules may be used to determine whether or not seminoma is present. Note, that there are many ways available to the skilled artisan to confirm that a tumor sample is a seminoma, and these need not be reiterated here.

It will also be seen from the examples that the invention embraces the use of the sequences in expression vectors, which may be used to transform or to transfect host cells and cell lines, be these prokaryotic (e.g., E. coli), or eukaryotic (e.g., human cells, rodent cells or simian cells, preferably CHO or COS cells). The expression vectors require that the pertinent sequence, i.e., those described supra, be operably linked to a promoter. The expression vector may include, e.g., a sequence encoding one or more HLA molecules. In a situation where the vector contains both coding sequences, it can be used to transform or transfect a cell which does not normally express either one. The tumor rejection antigen precursor coding sequence may be used alone, when, e.g., the host cell already expresses HLA-molecules. The particular host cell which is suitable for expressing the sequences described herein include, e.g., prokaryotic or eukaryotic cells, such as E. coli, human, CHO, COS cells or insect cells. The particular human cell line selected for expression of the sequences may depend on the needs of the experimental protocol and can be determined readily by one of skill in the art without further elaboration.

Another aspect of this invention is the isolation of a genomic DNA (gDNA) which encodes a protein having the amino acid sequence encoded by a nucleic acid molecule having the sequence set forth in SEQ ID NO: 9, 18, 21, 23, or 25. Such a gDNA may be identified and isolated using well-known methods in the art. For example MAGE-C1 specific probes derived from SEQ ID NO: 9 or MAGE-C2 specific probes derived from SEQ ID NO: 18 may be used to screen a genomic DNA library prepared from, e.g., LB373-MEL cells. Those of ordinary skill in the art will be able to determine from sequence analysis those sequences which are specific for MAGE-C1 and/or MAGE-C2. Likewise, MAGE-C3 specific probes, MAGE-B5 specific probes and MAGE-B6 specific probes derived respectively from SEQ ID NO: 21, 23, or 25 may also be used to screen a genomic DNA library to identify and isolate gDNA which encode proteins having the amino acid sequence encoded by a nucleic acid molecule having a sequence set forth in SEQ ID NO: 21, 22, or 23. It is also possible using techniques well known in the art to determine the chromosome where such a gDNA is located, see, e.g., PCT/US95/02203 incorporated in its entirety by reference.

MAGE-C1 specific, MAGE-C2 specific, MAGE-C3 specific, MAGE-B5 specific and MAGE-B6 specific oligonucleotides derived respectively from SEQ ID NO: 9, 18, 21,23, or 25 may also be useful in kits for PCR assays to amplify and thus detect MAGE-C3, MAGE-B5 and MAGE-B6 nucleic acid molecules in a sample. One of skill in the art can readily determine and generate oligonucleotides specific for each of the nucleic acid molecules of this invention that are useful for detection and analysis.

Another embodiment of this invention is an expression kit, which enables the artisan to prepare a desired expression vector or vectors. Such expression kits include at least separate portions of each of the previously discussed coding sequences, e.g., a vector such as a bacterial plasmid, a cosmid or a viral vector which comprises a promoter (DePlaen et al., Proc. Natl. Acad. Sci. 85:2274-2278 (1988), Grosveld et al., Gene 10:6715-6732 (1982), and Bates et al., Gene 26:137-146 (1983) incorporated in their entirety by reference, any of the HLA coding sequences, such as those set forth in Zemmour and Parham, Immunogenetics 37:239-250 (1993), or a MAGE-C1, MAGE-C2, MAGE-C3, MAGE-B5 or MAGE-B6 coding sequence, or both an HLA and a MAGE-C1, MAGE-C2, MAGE-C3, MAGE-B5, or MAGE-B6 coding sequence. Other components, such as, e.g., resistance markers, enhancers or inducible promoters which are known in the art may be added, as desired.

To distinguish the nucleic acid molecules and the TRAPs and TRAs of this invention from the previously described MAGE, BAGE, and GAGE materials, the invention shall be referred to as the MAGE-C1 gene, MAGE-C2 gene, MAGE-C3 gene, MAGE-B5 gene and MAGE-B6 gene, and as the MAGE-C1 TRAP and TRAs, MAGE-C2 TRAP and TRAs, MAGE-C3 TRAP and TRAs, MAGE-B5 TRAP and TRAs, and MAGE-B6 TRAP and TRAs. Hence, whenever MAGE-C1, MAGE-C2, MAGE-C3, MAGE-B5, or MAGE-B6 is used herein, it refers to the tumor rejection antigen precursors, and their derived TRAs, which are encoded for by the previously unknown nucleic acid sequences. “MAGE-C3 coding sequence”, “MAGE-B5 coding sequence” and similar terms, are used to describe the nucleic acid molecules themselves.

The invention as described herein has a number of uses, some of which are described herein. First, the invention permits the artisan to diagnose a disorder characterized by expression of the MAGE-C1, MAGE-C2 MAGE-C3, MAGE-B5 or MAGE-C6 messenger RNAs and the MAGE-C1, MAGE-C2, MAGE-C3, MAGE-B5 or MAGE-B6 TRAPs and TRAs. The methods involve determining the expression of mRNAs from the MAGE-C1, MAGE-C2, MAGE-C3, MAGE-B5 or MAGE-B6 nucleic acid molecules and related molecules, and/or the presence of TRAs derived from the TRAP encoded by MAGE-C1, MAGE-C2, MAGE-C3, MAGE-B5, or MAGE-B6 and related nucleic acid molecules. In the former situation, such determinations can be carried out via any standard nucleic acid determination assay, including the polymerase chain reaction, or assaying a sample of interest with labeled hybridization probes. In the latter situation, TRAP and TRA may be detected by assaying for the TRAP or TRA alone or assaying for complexes of TRA and HLA, using binding partners such as, e.g., antibodies. Another embodiment of this invention is a method to detect the presence of cytolytic T cells specific for complexes of an HLA molecule and a peptide derived from a protein, wherein the protein has an amino acid sequence encoded by an isolated nucleic acid molecule having a sequence set forth by SEQ ID NO: 18, SEQ ID NO: 21, SEQ ID NO: 23 or SEQ ID NO: 25, in a CTL-containing sample, wherein the method comprises contacting the CTL-containing sample with cells that present the complexes on their surface, and determining (i) proliferation of cytolytic T cells, or (ii) lysis of cells presenting said complexes. Proliferation or lysis indicates the cytolytic T cells are present in the sample. CTL proliferation may be detected by assaying TNF release or the release of a radiolabelled substance, such as ⁵¹Cr, as described, e.g., in PCT/US95/02203 incorporated in its entirety by reference. In addition, CTL may be detected by ELISPOT analysis as per Schmitt et al., J. Immunol. Meth., 210:167-179 (1997) and Lalvani et al. J. Exp. Med., 186:859 (1997), both of which are incorporated by reference or by FACS analysis of fluorogenic tetramer complexes of MHC class I/peptide (Dunbar et al. Current Biology, 8:713-716 (1998)).

The isolation of nucleic acid molecules of these members of the MAGE-C and MAGE-B families also makes it possible to isolate the TRAP molecules themselves, especially TRAP molecules consisting of the amino acid sequence encoded by SEQ ID NO: 9 or SEQ ID NO: 18, SEQ ID NO: 21, SEQ ID NO: 23 or SEQ ID NO: 25. The isolation of these MAGE-C and MAGE-B nucleic acid molecules also makes it possible to identify TRAs that are unique to MAGE-C1, C2, C3, B5 or B6 discussed in more detail infra.

Further, the polypeptide having the amino acid sequence encoded by nucleotide sequence 257-3682 of SEQ ID NO: 9, the polypeptide having the amino acid sequence encoded by nucleotide sequence 330-1449 of SEQ ID NO: 18 and polypeptides derived therefrom are also part of this invention. Also an embodiment of this invention is a polypeptide which comprises the polypeptide encoded by the sequence set forth in nucleotides 3761-4801 (SEQ ID NO: 21) of the PAC clone 232G24, preferably the polypeptide consists of the amino acid sequence encoded by nucleotides 3761-4801 of PAC clone 232G24 or peptides derived therefrom. Another embodiment of this invention is a polypeptide comprising the sequence set forth by nucleotides 124185-123358 (SEQ ID NO: 23) of the BAC clone 46604 of Bacterial artificial Chromosome library RPCI11 available under accession nu. AC005297 from the Human Genome Sequencing Center at Baylor College of Medicine (Houston, Tex.) and polypeptides derived therefrom. Still a further object of this invention is a polypeptide comprising the amino acid sequence encoded by nucleotides 147640-146417 (SEQ ID NO: 25) of the BAC clone 46604 of the Bacterial Artificial Chromosome library RPCI11 produced by the Human Genome Sequencing Center at the Baylor College of Medicine and can be obtained under accession number AC005297. These polypeptides alone or in combination with other polypeptides from other TRAP molecules, for example, may be combined with materials such as adjuvants which are well-known in the art see, e.g., U.S. Pat. No. 5,057,540 to Kensil et al., incorporated by reference or PCT application PCT/US92/03579 to Scott et al., also incorporated by reference to produce vaccines which will be useful in treating disorders characterized by expression of the molecules.

Polytopes are groups of 2 or more potentially immunogenic or immune stimulating peptides, which can be joined together in various ways, to determine if this type of molecule will stimulate and/or provoke an immune response. Peptides of the MAGE-C and MAGE-B TRAPs of this invention may also be combined to form polytopes and used to produce vaccines useful in treating disorders characterized by expression of one or more of the TRAs. Preferably, the polytopes comprise a plurality of peptides derived from MAGE-C1, MAGE-C2, MAGE-C3, MAGE-B5, and MAGE-B6 TRAPs.

Peptides derived from the polypeptide having the amino acid sequence encoded by nucleotide sequence 257-3682 of SEQ ID NO: 9 and the polypeptide having the amino acid sequence encoded by nucleotide sequence 330-1449 of SEQ ID NO: 18 which are presented by MHC molecules and recognized by CTL may be combined with peptides from other tumor rejection antigen precursors to form “polytopes.” Exemplary peptides include those listed in U.S. patent application Ser. Nos. 08/672,351; 08/718,964 now U.S. Pat. No. 5,811,519; Ser. No. 08/487,135 now U.S. Pat. No. 5,821,122, Ser. Nos. 08/530,569 and 08/880,963 all of which are incorporated by reference.

Additional peptides which can be used are those described in the following references, all of which are incorporated by reference: U.S. Pat. Nos. 5,405,940; 5,487,974; 5,519,117; 5,530,096; 5,554,506; 5,554,724; 5,558,995; 5,585,461; 5,589,334; 5,648,226; and 5,683,886; PCT International Publication Nos. 92/20356; 94/20356; 96/10577; 96/21673; 97/10837; 97/26535; and 97/31017, as well as pending U.S. application Ser. No. 08/713,354.

These peptides can be joined together directly, or via the use of flanking sequences. See Thomson et al., Proc. Natl. Acad. Sci. USA, 92(13):5845-5849(1995) (incorporated by reference), teaching the direct linkage of relevant epitopic sequences. The use of polytopes as vaccines is well known. See, e.g., Gilbert et al., Nat. Biotechnol., 15(12):1280-1284 (1997); Thomson et al., supra; Thomson et al., J. Immunol., 157(2):822-826 (1996); Tam et al., J. Exp. Med. 171(1):299-306 (1990), all of which are incorporated by reference. Tam et al., in particular, shows that polytopes, when used in a mouse model, are useful in generating both antibody and protective immunity. Further, the reference shows that the polytopes, when digested, yield peptides which can be and are presented by MHCs. Tam et al. shows this by demonstrating recognition of individual epitopes processed from polytope “strings,” via CTLs. This approach can be used, e.g., in determining how many epitopes can be joined in a polytope, and still provoke recognition and also to determine the efficacy of different combinations of epitopes. Different combinations maybe “tailor-made” for patients expressing particular subsets of tumor rejection antigens. These polytopes can be introduced as polypeptide structures, or via the use of nucleic acid delivery systems. To elaborate, the art has many different ways available to introduce DNA encoding an individual epitope, or a polytope such as is discussed supra. See, e.g., Allsopp et al., Eur. J. Immunol. 26(8):1951-1959 (1996), incorporated by reference. 1 Adenovirus, pox virus, Ty-virus like particles, plasmids, bacteria, etc., can be used. One can test these systems in mouse models to determine which system seems most appropriate for a given, parallel situation in humans. They can also be tested in human clinical trials.

In addition, vaccines can be prepared from cells, such as non-proliferative cancer cells, non-proliferative transfectants, etcetera, which present the TRA/HLA complexes on their surface. In all cases where cells are used as a vaccine, the cells may be transfectants having been transfected with coding sequences for one or both of the components necessary to provide a CTL response, i.e., TRAP, TRA, and HLA molecules using techniques which are well-known in the art see, e.g., PCT/US95/02203 and Zemmour supra for sequence of various HLA molecules. Alternatively, the cells may express both HLA and TRAP/TRA molecules without transfection. Further, the TRAP molecules, their associated TRAs, as well as complexes of TRA and HLA, may be used to produce antibodies, using standard techniques well known in the art.

When “disorder” is used herein, it refers to any pathological condition where the tumor rejection antigen precursor is expressed. An example of such a disorder is cancer, e.g., seminoma, bladder transitional-cell carcinoma, head-and-neck squamous-cell carcinoma, breast carcinoma, sarcoma, cutaneous melanoma, lung carcinoma (NSCLC): seminoma in particular.

Therapeutic approaches based upon the disclosure herein are premised on a response by a subject's immune system, leading to lysis of HLA/TRA presenting cells. One such approach is the administration of CTLs which are specific to an HLA/TRA complex to a subject having abnormal cells of the phenotype at issue. It is within the skill of the artisan to develop such CTLs in vitro see, e.g., Herin et al. supra. For example, a sample of cells, such as blood cells, are contacted to a target cell presenting an HLA/TRA complex and capable of provoking a specific CTL to proliferate. The target cell can be a transfectant, such as a COS cell transfected with and expressing a particular HLA and TRAP as described supra. These transfectants present the desired complex on their surface and, when combined with a CTL of interest, stimulate its proliferation. COS cells, such as those used herein are widely available, as are other suitable host cells including but not being limited to, human cells, CHO cells, Spodopitera furjiperda, E. Coli, Bacillus, and so forth.

One therapeutic methodology is referred to as adoptive transfer (Greenberg, J. Immunol. 136(5): 1917 (1986); Riddel et al., Science 257: 238 (7-10-92); Lynch et al., Eur. J. Immunol. 21: 1403-1410 (1991); Kastetal., Cell 59: 603-614 (11-17-89)). In adoptive transfer, cells presenting the desired HLA/TRA complex are combined with CTLs leading to proliferation of the CTLs which are specific for that complex. The proliferated CTLs are then administered to a subject with a cellular abnormality which is characterized by certain of the abnormal cells presenting the particular complex. The CTLs then lyse the abnormal cells, thereby achieving the desired therapeutic goal.

The foregoing therapy assumes that at least some of the subjects abnormal cells present the relevant HLA/TRA complex. This can be determined easily, as the art is very familiar with methods for identifying cells which present a particular HLA molecule, as well as how to identify cells expressing DNA of the pertinent sequences, in this case MAGE-C1, MAGE-C2, MAGE-C3, MAGE-B5, or MAGE-B6 and related sequences. If the abnormal cells of the patient present the relevant HLA/TRA complex then the patient is an appropriate candidate for the therapeutic approaches set forth supra.

Adoptive transfer is not the only form of therapy that is available in accordance with the invention. CTLs can also be provoked in vivo, using a number of approaches. One approach, i.e., the use of non-proliferative cells expressing the complex as a vaccine, has been elaborated upon supra. The cells used in this approach may be those that normally express the complex, such as irradiated seminoma cells or irradiated cells transfected with one or both of the genes necessary for presentation of the complex. Chen et al., Proc. Natl. Acad. Sci. USA 88:110-114 (January 1991) exemplifies this approach, showing the use of transfected cells expressing HPV E7 peptides in a therapeutic regime. Various cell types may be used. Similarly, vectors, such as viral or bacterial vectors, carrying a nucleic acid molecule encoding either an HLA or a TRAP or TRA, or combination thereof, may be used. In these systems, the nucleic acid molecule is carried by, e.g., a Vaccinia virus or the bacteria BCG, which “infect” host cells. The infected cells present the HLA/TRA complex and are recognized by autologous CTLs, which then proliferate. CTLs can also be provoked in vivo by combining the TRA or the TRAP itself with an adjuvant to facilitate incorporation into HLA presenting cells. The cells present the HLA/peptide complex of interest by further processing the TRAP to yield the peptide partner of the HLA molecule. Alternatively, the cells may present the TRA without the need for further processing. See, e.g., Braciale, T. J. and Braciale, V. L., Immunology Today 12:124-129 (1991); T. Elliot, Immunology Today 12:386-388 (1991), and: Madelboim et al., Nature 369:67-71(1994). Also, a feature of this invention are isolated peptides derived from the MAGE-C1 TRAP, MAGE-C2 TRAP, MAGE-C3 TRAP, MAGE-B5 TRAP, or MAGE-B6 TRAP, which conform to the rules for presentation by MHC molecules. For example, in PCT application No. PCT/US93/07421, incorporated by reference herein, several motifs are described as being associated with different MHC molecules. These motifs, incorporated by reference herein, as well as those taught by, e.g., Falk et al., Nature 351:290-296 (1991); Engelhard, Ann. Rev. Immunol 12:181-207 (1994); Ruppert et al., Cell 74:929-937 (1993); Rotzschke et al., Nature 348:252-254 (1990); Bjorkman et al., Nature 329:512-518 (1987) and Traversari et al., J. Exp. Med. 176:1453-1457 (1992) all of which are incorporated by reference, serve as a basis for identifying appropriate peptides obtainable or derivable from the MAGE-C and MAGE-B amino acid sequences and the nucleotide sequences which encode the proteins disclosed herein. In another aspect of the invention these peptides may be used alone, or in mixtures, to stimulate CTL proliferation. These peptides are also useful in vaccines.

It is well established that the blood of individuals afflicted with tumors frequently contains cytolytic T cells (“CTLs”) which recognize complexes of MHC molecules and presented peptides. See, e.g., Robbins et al., Canc. Res. 54:3124-3126 (1994); Topolian et al., J. Immunol. 142:3714-3725 (1989); Coulie et al., Int. J. Cancer 50:289-297 (1992), all of which are incorporated by reference. Also, note Kawakami et al., J. Ex . Med. 180:347-352 (1994); Hom et al., J. Immunother. 10:153-164 (1991), Darrowet al, J. Immunol. 142(9):3329-3335 (1989); Slovin etal., J. Immunol. 137(9):3042-3048 (1986), all of which are incorporated by reference. These papers all establish the usefulness of a CTL proliferation assay to diagnose possible cancer.

In general, a patient will only have CTLs which recognize and proliferate in response to contacting target cells presenting particular complexes of TRA and HLA only if at least some of the patient's own cells are also expressing that particular complex. If one takes a peripheral blood lymphocyte (PBL) containing sample from a patient suspected of having abnormal cells, e.g., tumor cells, and contacts that CTL-containing sample with a target cell which presents complexes of a relevant MHC molecule and a MAGE-C1, MAGE-C2, MAGE-C3, MAGE-B5, or MAGE-B6 derived peptide one will only see proliferation of CTLs which are specific for that complex. Thus proliferation of CTLs in the patient's PBL sample will indicate that the patient possibly has tumor cells which express that particular HLA/TRA complex. The target cells may be cells which normally present the MHC molecule in question or may be cells which have been transfected with an HLA coding sequence. The target cells may conceivably be tumor cells, or normal cells.

One embodiment of the invention involves mixing a target cell sample with (1) a peptide or mix of peptides which are derived from MAGE-C1, a MAGE-C2, MAGE-C3, MAGE-B5, or MAGE-B6 TRAPs and presented by the target cell MHC molecules and (2) a PBL sample of the subject under evaluation. The mixture is then tested for CTL proliferation. Various methods of determining CTL proliferation are known in the art, e.g., TNF release assays, and ⁵¹Cr release assays see, e.g.,

The peptide or peptides of this invention may also be combined with one or more adjuvants to stimulate a more pronounced CTL response. Exemplary of such adjuvants are saponins and their derivatives, such as those disclosed by U.S. Pat. No. 5,057,540 to Kensil et al., incorporated by reference or PCT application PCT/US92/03579 to Scott et al., also incorporated by reference. Of course, standard adjuvants, such as Freund's complete adjuvant, or Freund's incomplete adjuvant, may also be used.

Other aspects of the invention will be clear to the skilled artisan and need not be repeated here.

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

### EXAMPLE 1

### Generation of Difference Products (DP) For Tumor LB373-MEL and Testis

A cDNA library enriched for sequences present only in the cell type of interest, a “tester” cell, and not present in another cell type, a “driver” cell, was generated essentially as described by Hubank and Schatz, Nucl. Acids. Res. 22:5640-5648 (1994) incorporated herein in its entirety by reference. Briefly, total RNA was prepared from tester cells and driver cells. Herein the tester cells were melanoma cells LB373-MEL and the driver cells were normal skin cells. Poly-A+ RNA was isolated from total RNA using oligo-dT columns using techniques well known in the art. The poly-A+ RNA was then reverse transcribed to produce cDNA. The cDNA was digested with restriction enzyme DpnII, which cuts DNA at GATC sites, to generate short fragments of double stranded DNA with 5′-GATC overhangs. Double-stranded DNA adapters with a 5′-GATC overhangs (R-Bgl adaptor which is composed of annealed R-Bgl-12 and R-Bgl 24 oligonucleotide SEQ ID NO: 2 and SEQ ID NO: 11 respectively) were ligated to the DpnII digested cDNA prepared from the tester and driver cells. The adaptor-ligated cDNA was subsequently amplified by the well-known polymerase chain reaction (PCR). The amplified product is a “representation” of the tester and the driver, respectively. Both tester and driver representations were digested with DpnII. Digested tester was ligated to new adaptor molecules (J-Bgl adaptor which is composed of annealed J-Bgl-12 and J-Bgl-24 oligonucleotide SEQ ID NO: 3 and SEQ ID NO: 12 respectively). A first round of subtractive hybridization was then performed by mixing in 100/1 proportions the digested driver cDNA with the tester cDNA ligated to the J-Bgl adapters. The mixed driver and tester cDNA sample was denatured at 98° C. for 5 min and then incubated at 67° C. for 20 hours to rehybridize the denatured sample. This resulted in a mixture of hybrid double-stranded cDNAs. The hybrid cDNAs were of three types. One hybrid type constituted two tester cDNA molecules which represented nucleotide sequences unique to the tester cells, a second hybrid type constituted two driver cDNA molecules and a third hybrid type constituted one tester cDNA molecule and one driver cDNA molecule. After hybridization, the sample was PCR amplified using a single stranded J-Bgl adaptor, J-Bgl-24 SEQ ID NO: 12. Hybrid cDNAs composed of two driver cDNA molecules were not amplified, because they did not comprise the J-Bgl adaptor. Hybrid cDNAs constituted by one tester cDNA molecule and one driver cDNA molecule were only amplified linearly. Only double stranded cDNA consisting of two tester cDNA molecules were amplified exponentially.

After 10 cycles of PCR amplification as described supra, the sample was treated with Mung Bean Nuclease (which digests specifically the single stranded DNA produced by the linear amplification), then subjected to 18 additional PCR cycles.

The resulting enriched product was designated difference product 1 (DP1). DP1-Testis[-HLLK] (testis=tester cDNA and HLLK=driver cDNA, a mix of normal heart, lung, liver and kidney cDNA) and DP1-LB373 [-skin] were both generated.

J-Bgl adapters on DP1 were changed for N-Bgl-12/24 adapters (N-Bgl-12: 5′GATCTTCCCTCG-3′; N-Bgl-24: 5′-AGGCAACTGTGCTATCCGAGGGAA-3′), i.e., annealed N-Bgl-12 and N-Bgl-24 oligonucleotides, SEQ ID NO: 4 and SEQ ID NO: 13 and the process of subtractive hybridization and selective amplification repeated to generate the second difference products (except that annealing and extension in PCR reactions were performed at 72° C.). Tester to driver ratios were of 1/800 to generate DP2.Testis[-HLLK], but of 1/100 to generate DP2.LB373[-skin]. A third difference product DP3.Testis [-HLLK] was generated by repeating the process with J-Bgl ligated DP2.Testis[-HLLK] as tester and HLLK representation as driver, with final amplification performed of 22 cycles.

### EXAMPLE 2

### Search For Sequences Common to DP2.LB373[-skin] and DP3.Testis[-HLLK]

Many known tumor antigens are encoded by genes that are expressed only in tumors and in testis. By searching for sequences that were common to both DP3.Testis[-HLLK] (representing nucleic acid sequences unique to testis cells) and DP2.LB373[-skin] (representing nucleic acid sequences unique to melanoma cells), as described supra nucleic acid sequences were identified that were expressed only in testis and tumor cells that encode previously unidentified tumor antigens.

To clone DP3.Testis[-HLLK] DNA, DP3.Testis[-HLLK] was digested with DpnII and the digested DNA was ligated to BamHI digests of the commercially available plasmid pTZ18R. The bacteria, DH5αF′IQ (commercially available), was electroporated with ligated DNA. The electroporated bacteria were selected and screened by colony hybridization with a probe produced by labeling DP2.LB373 [-skin] with random primers, Klenow DNA polymerase and α-³²P-dCTP.

Plasmids from transformants which hybridized to the DP2.LB373 [-skin] probe were isolated and their inserts analyzed. One clone containing a 283 bp insert was purified and sequenced using techniques well known in the art. The sequence of the 283bp insert shared partial homology with the MAGE gene family. Maximum homology (74%) was obtained with a 147 nucleotide sequence, corresponding to nucleotides 9895 to 10041 of MAGE-4a cDNA, as predicted from the MAGE 4a genomic DNA (Genbank accession no. U 10687), incorporated herein by reference. These data suggested that the 283 bp insert was a portion of a previously unidentified MAGE family member. This family member was designated MAGE-C1.

### EXAMPLE 3

### Complete MAGE-C1 cDNA

To obtain the complete MAGE-C1 cDNA, a cDNA library, prepared from LB373-MEL RNA and subcloned into pcDNAI/Amp, was screened. The cDNA library was prepared as follows.

Total RNA was extracted from LB373-MEL cells by the guanidine-isothiocyanate procedure (Davis L. G., M. D., Dibner and J. F. Battery, Basic Methods in Molecular Biology, Elsevier, N.Y., pp. 130-135 (1986)). Poly-A+ RNA was purified on oligo-dT columns (Pharmacia) and converted to cDNA using an oligo-dT (Notl, EcoRI) primer SEQ ID NO: 5. The cDNA was ligated to BstXI adaptors (SEQ ID NO: 6), digested with NotI and ligated with BstXI and NotI digested commercially available expression vector pcDNAI/Amp using methods well known in the art. Top 10F′ Escherichia coli bacteria were electroporated with the ligated recombinant plasmids and transformants selected with ampicillin (50 μg/ml). The library was screened with a ³² P-radiolabelled probe derived from the 283 bp insert isolated supra.

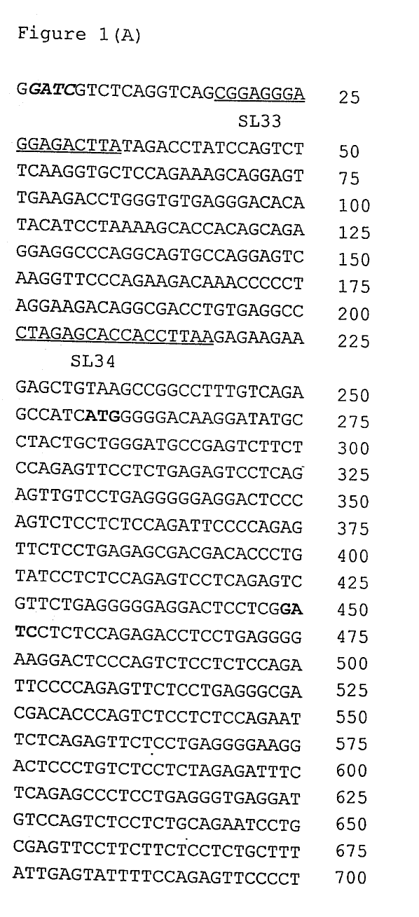

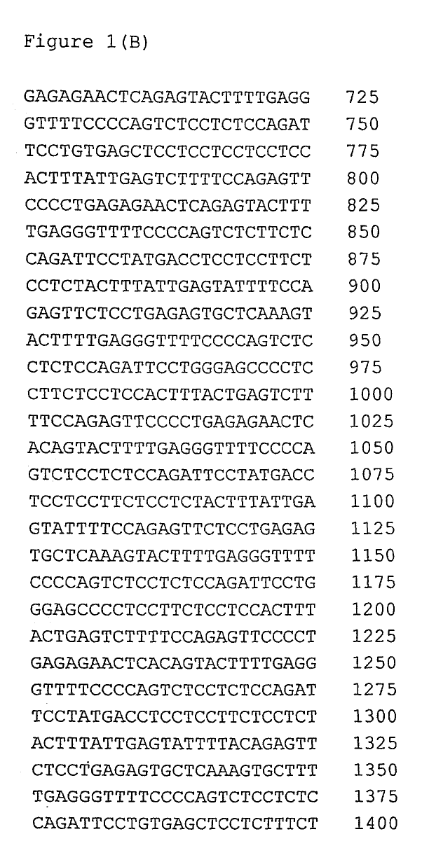

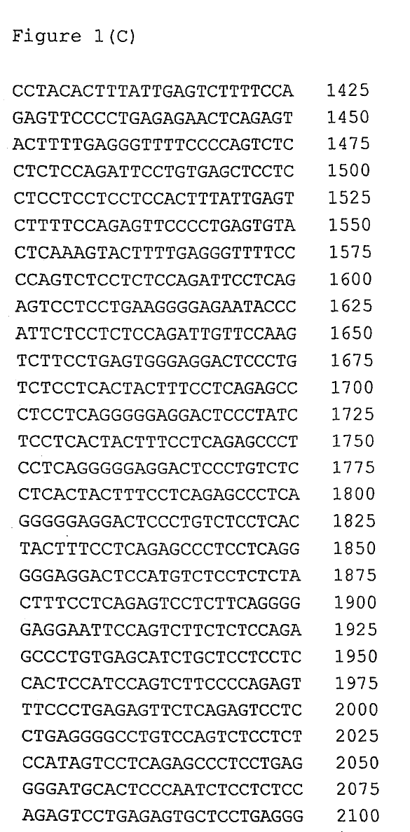

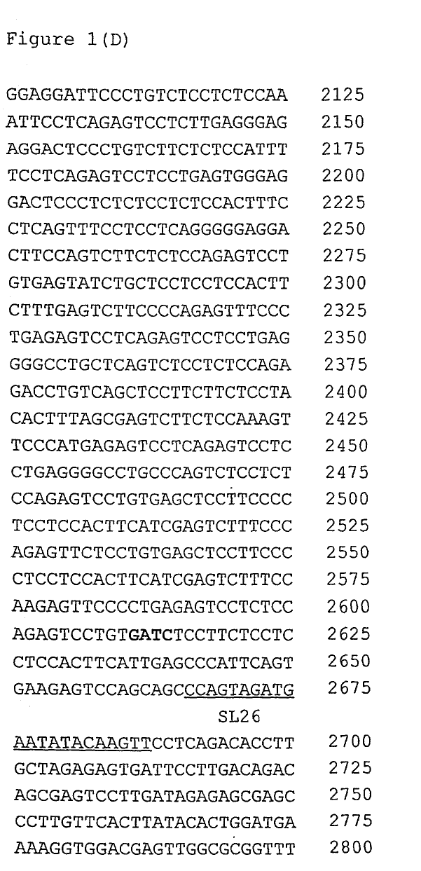

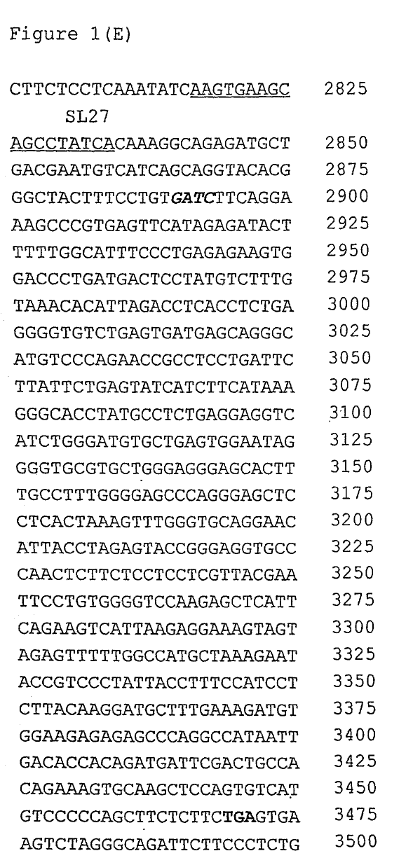

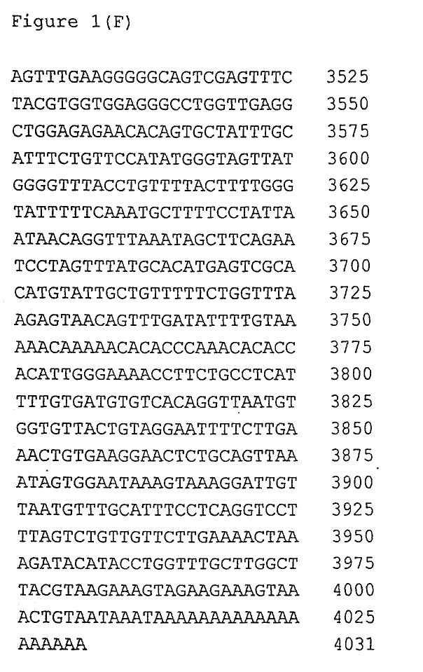

Bacterial transformants were screened for MAGE-C1 sequences by using methods well-known in the art. Briefly, approximately 140,000 bacteria were plated on nylon membrane filters. Duplicate nylon membrane filters were made and treated to denature and fix the bacterial DNA. A 168 bp MAGE-C1 specific probe was generated by RT-PCR (reverse transcription-PCR) using LB373-MEL RNA as template, and MAGE-C1 specific primers, i.e., sense primer SL26: 5′ CCAGTAGATGAATATACAAGTT-3′ which corresponds to nucleotides (nt) 2666 to nt 2687 of SEQ ID NO: 1 and antisense primer SL27: 5′-GATAGGCTGCTTCACTT-3′, which is the complementary sequence of nt 2817 to nt 2833 of SEQ ID NO: 1. This 168 bp MAGE-C1 PCR product, which corresponds to nt 2666 to 2833 of SEQ ID NO: 1, was purified on a sepharose CL-6B column, then labeled using random primers, Klenow DNA polymerase and α-³²P-dCTP as described supra (Example 1). The treated duplicate membrane filters were hybridized with the MAGE-C1 specific probe (500,000 cpm/ml; overnight incubation at 65° C. in 5× SSC, 0.1% SDS 5× Denhardt's reagent), then washed in stringent conditions, and autoradiographed for 70 hours at room temperature. Stringent conditions as described herein refers to 0.1× to 0.5× SSC, 0.1% SDS at 65° C. for 20 min. Two colonies were identified which hybridized to the MAGE-C1 probe. The colonies were purified and screened once again to verify that they hybridized to the probe. Plasmids were isolated from these colonies and their inserts sequenced and analyzed using methods which were well-known in the art. One clone was selected and the MAGE-C1 cDNA inserted analyzed in detail. The analyzed clone contained a MAGE-C1 cDNA molecule 4031 bp long (FIG. 1) SEQ ID NO: 1. An open reading frame (ORF) runs almost through the entire cDNA with a first ATG, located at nt 257, in accordance with the known Kozak rule, and a stop codon at nt 3473. The ORF encodes a putative protein of 1072 amino acids.

Alignment with the MAGE-A1 cDNA revealed significant homologies between the MAGE-C1 cDNA (SEQ ID NO: 1) and MAGE-A1 exons 2 and 3. The open reading frame of MAGE-C1, however, is about 2 kb longer than that of MAGE A1, most of the difference being accounted for by a large repetitive sequence.

### EXAMPLE 4

### MAGE-C1 Expression

Sense primer SL33 (5′-CGGAGGGAGGAGACTTA-3′) nt 18-34 of SEQ ID NO: 1 and antisense primer SL34 (5′-TTAAGGTGGTGCTCTAGG-3′) which is complementary to nt 200-217 of SEQ ID NO: 1 are shown in FIG. 1. These primers are located in different exons, as determined by the different sizes of PCR products from cDNAs (202 bp) or genomic DNAs (approximately 1.1 kb) prepared from normal tissue and tumor cells. The expression pattern of the MAGE-C1 messenger RNA was determined by standard RT-PCR analysis of normal tissue and tumor samples. The data indicate that MAGE-C1 expression is not detected in the normal tissues tested (Table 1), with the exception of testis. Among tumor cell samples, MAGE-C1 expression is frequently detected in melanoma (46%), seminoma (100%), bladder transitional-cell carcinoma (18%), breast carcinoma (16%) and non-small cell lung carcinoma (16%). It is also detected in a significant fraction of sarcoma, head and neck carcinoma, and prostate adenocarcinoma (Table 2).

### EXAMPLE 5

### Northern Blot Analysis

10 μg total RNA extracted by the guanidine-isothiocyanate procedure (Davis et al., Basic Methods in Molecular Biology, Elsevier, N.Y., pp.130-135 (1986) were separated by formaldehyde agarose gel electrophoresis, transferred to a nylon membrane by capillary transfer and fixed by UV irradiation. Hybridization to the MAGE-C1 1.3kb Xbal-EcoRI probe corresponding to nucleotide 589 to 1904 of SEQ. ID. NO: 1 (radiolabeled with [α-³²P]dCTP) was performed overnight at 60° C. in 10% dextran sulfate, 1M NaCl, 1% SDS and 100 μg/ml denatured salmon sperm DNA. The membrane was washed consecutively in 2× SSC, 0.1% SDS for 20 min at room temperature, in 2× SSC, 0.1% SDS for 20 min at 60° C., and finally in 0.2× SSC, 0.1% SDS for 5 min at 60° C. Autoradiography was performed for 7 days using BioMax MS film (Kodak). The same membrane was-hybridized to a β-actin specific probe in identical conditions, except washing was performed twice for 10 min in 2× SSC at room temperature and autoradiography performed overnight. A MAGE-C1 messenger species migrating around 4 kb in total RNA from normal testis and some tumor cell lines was observed. No MAGE-C1 messenger species were detected in total RNA from normal lung.

### EXAMPLE 6

### Structure of the MAGE-C1 cDNA

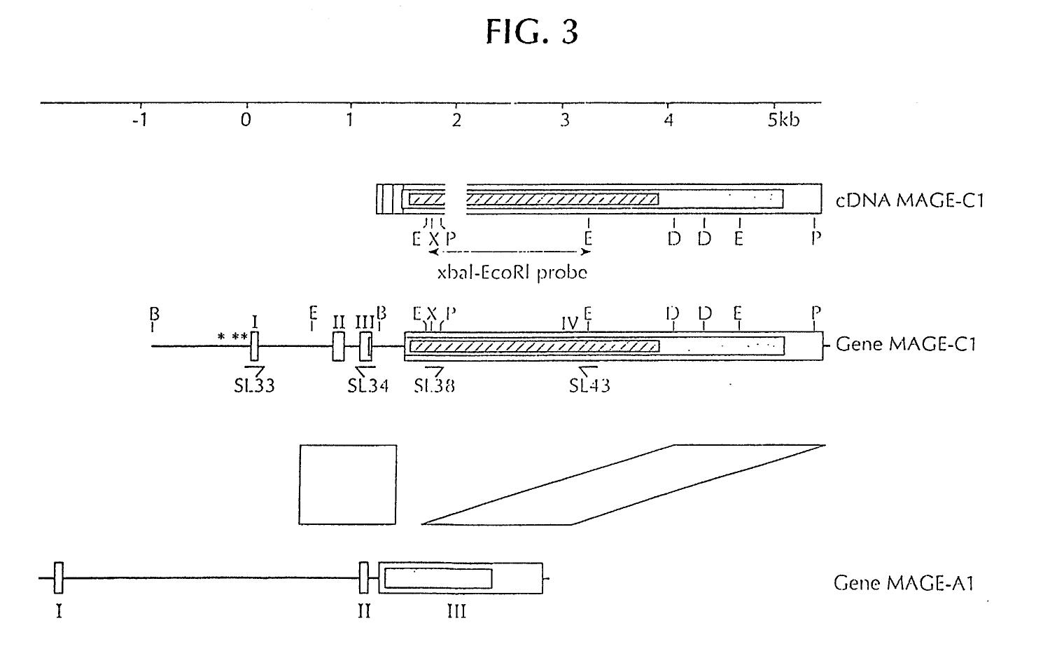

Sequencing and alignment of SEQ ID NO: 1 (FIG. 2 and FIG. 3) revealed that the MAGE-C1 cDNA is homologous to MAGE-A1 (Van der Bruggen et al., Science 254: 1643 (1991)) only in its 3′ third. Except for another short stretch of homology to the second exon of MAGE-A1, MAGE-C1 is composed of sequences unrelated to MAGE family or to any sequence reported in databanks. Compared to other MAGE cDNAs, MAGE-C1 contains an approximately 2.4 kb insertion represented in FIG. 3 by a large hatched box, which comprises 3 types of tandemly repeated sequences: 42 bp-repeats, 63 bp-repeats, and 48 bp-repeats.

### EXAMPLE 7

### Southern Blot Analysis

Southern blots prepared with several genomic DNAs from melanoma cell lines LB373-MEL, SK29-MEL, and LB33. A-1, (Coulie et al., J. Exp. Med. 180:35-42 (1994); Coulie et al., Proc. Natl. Acad. Sci. USA 92:7976-7980 (1995); Lehmann et al. Eur. J. Immunol. 25:340-347 (1995)), were hybridized with a 1.3 kb Xbal-EcoRI cDNA probe derived from SEQ ID NO: 1, which contains most of the insertion that distinguishes cDNA clone MAGE-C1 from other MAGE cDNAs. Ten μg genomic DNA digested with a restriction enzyme were separated by agarose gel electrophoresis, transferred to nylon membranes by the capillary transfer method and fixed by UV irradiation as described (Sambrook et al., Molecular Cloning. A Laboratory Manual, N.Y. Cold Spring Harbor Laboratory Press, pp. 9.31-9.58, incorporated here by reference). Hybridization to the [α-³²P]dCTP radiolabeled MAGE-C1 1.3 kb XbaI-EcoRI probe was performed in 5× SSC, 5× Denhardt's, 0.1% SDS and 100 μg/ml denatured salmon sperm DNA for 12 to 24 hours at 68° C. Membranes were washed consecutively in 2× SSC, 0.1% SDS for 20 min at room temperature, in 2× SSC, 0.1% SDS for 20 min at 68° C., and in 0.2× SSC, 0.1% SDS for 20 min at 68° C. Autoradiography was performed for 3 days using BioMax MS film (Kodak).

A single hybridizing band was present in DNA from the SK29 melanoma line digested with 5 distinct restriction enzymes, suggesting that MAGE-C1 is the only gene of its type in the MAGE-family. However, Pstl digested DNAs isolated from peripheral blood lymphocytes of 11 male patients contain each a unique MAGE-C1 band, but of different sizes, suggesting the existence of allelic polymorphism in gene MAGE-C1. EcoRI digested DNAs from LB373-MEL and LB33-MEL.A-1 contain a unique MAGE-C1 band of identical size (see FIG. 3 for positions of probe and restriction sites).

### EXAMPLE 8

### Isolation of MAGE-C1 Gene

To isolate the MAGE-C1 gene, a cosmid library prepared with genomic DNA from melanoma line LB33-MEL.A-1 was screened. Genomic DNA from melanoma line LB33-MEL.A-1 was partially digested with Mbol and ligated to cosmid arms of vector c2RB as described (Lurquin, C. et al., Cell 58:293-303 (1989)) incorporated by reference]. The ligated DNA was packaged into λ phage heads (GIGAPACK, Strategene) and titrated on Escherichia coli ED8767. The library was represented by 40 groups of 70,000 independent cosmids. Each group was used to infect Ed8767 bacteria, and amplified in LB medium containing 50 μg/ml ampicillin. Aliquots of 16 hour-cultures were frozen, others were titrated to evaluate the amplification of the library (10⁵×), and the remainder of the cultures was further amplified and used to isolate total cosmid DNA, as described (De Plaen, Immunology Methods Manual Academic Press Ltd., 9.9: 691-718 (1997) incorporated by reference).

DNA extracted from 16 groups of approximately 70,000 independent cosmids was submitted to PCR amplification with MAGE-C1 primers. Twelve groups were found positive, and one of these was screened by colony hybridization with the Xbal-EcoRI probe. A positive cosmid, C7.2, was identified. Restriction analysis and Southern blot revealed that this cosmid contained an approximately 42 kb insert carrying 4 EcoRI fragments of 1, 1.4, 1.6, and 2 kb, respectively, and one BamHI fragment of 2 kb, which hybridized with a probe corresponding to the entire MAGE-C1 cDNA clone (SEQ ID NO: 1). Those 5 fragments were subcloned in phagemid pTZ19R and their nucleotide sequence was determined. Comparison of these sequences with the cDNA clone showed that MAGE-C1 is composed of four exons (FIG. 3). A 3,426 base pair open reading frame starts with an ATG located at the end of exon III, and runs through most part of exon IV. All repeated motifs are included in the latter but the length of this repetitive region was longer in the gDNA clone as compared to that found in the cDNA clone. Although the cDNA and genomic clones came from libraries of different origins (sublines of LB373-MEL and LB33-MEL.A-1 respectively), allelic variation could hardly explain this discrepancy, as demonstrated by Southern blot analysis with the Xbal-EcoRI probe. To confirm Southern analysis results, genomic DNA from both cell lines was amplified by PCR with primers SL38 (5′-GGCGACGACACCCAGT-3′) corresponding to nt 521 to 536 of SEQ ID NO: 1 and SL43 (5′-AGGAAAGTAGAGAGGAGACAT-3′) corresponding to nt 1862 to 1882 of SEQ ID NO: 1 and products of identical sizes were obtained. Partial sequencing of these PCR products showed no difference at the nucleotide level between the two cell lines, excluding the presence of a splice site in LB373-MEL cells, that is absent in LB33-MEL cells.

To determine if reverse transcription artifacts accounted for the differing lengths of the repetitive regions in the gDNA and cDNA clones, cDNA obtained from reverse transcription of total RNA was amplified by PCR using primers SL38 and SL43.

The Transcription in vitro Systems (Promega) was used to produce MAGE-C₁ RNA for the PCR amplification and cloning of MAGE-C1 repetitive region from cDNA. One μg HindIII digested pcDNAI/Amp containing MAGE-C1 cDNA clone was diluted to a final volume of 20 μl with 4 μl 5× SP6 buffer, 1 μl each NTP at 10 mM, 2 μl dithiotreitol at 0.1 M, 0.5 μl (20 Units) RNase inhibitor and 1 μl (15 units) SP6 RNA polymerase. A control reaction was set up where 5 μl [α-³²P]CTP (3000 Ci/mmol) were added to a mixture identical to the transcription mixture described above, except that only 2.4 μl of 0.1 mM CTP were used. The reactions were incubated at 37° C. for 1 hour. One μl (1 U) RQ1 DNase was added to the mixtures which were incubated again for 1 hour at 37° C. One tenth of the radiolabeled RNA was analyzed by electrophoresis on a formaldehyde agarose gel, the gel was dried and autoradiographed to confirm that only full length products were obtained. Non-radioactive RNA was phenol extracted, ethanol precipitated, and resuspended in 10 μl water. One μl RNA solution was reverse transcribed in the same conditions as total RNA (Weynants et al., Int. J. Cancer 56:826-829 (1994)), incorporated herein by reference). To exclude contamination with plasmid DNA, a control reaction was included where no MoMLV reverse transcriptase was added. 1/40 of the completed reactions were engaged in 37 PCR cycles with SL38 sense primer and SL43 anti-sense primer. PCR products were fractionated by agarose gel electrophoresis. No detectable product were detected in control reactions.

Sense primer SL38 (5′-GGCGACGACACCCAGT-3′) corresponding to nt 521 to 536 of SEQ ID NO: 1 and anti-sense primer SL43 (5′AGGAAAGTAGAGAGGAGACAT-3′) corresponding to nt 1862 to 1882 of SEQ ID NO: 1 were used to amplify cDNA (1/40 of reverse transcription product from 2 μg total RNA) or 500 ng genomic DNA from melanoma lines LB373-MEL and LB-33-MEL.A-1. PCR was performed in 50 μl final volume, with 5 μl 10× DynaZyme buffer, 1 μl each of 10 mM dNTP, 25 pmoles each primer and 2 units DynaZyme (FynnZymes Oy), for 30 (genomic DNA) or 37 (cDNA) cycles of 1 min at 94° C., 1 min at 65° C. and 2 min at 72° C.

PCR products were ligated to plasmid pCR3 using the Eukaryotic TA Cloning Kit (Invitrogen), and ligation products were electroporated in Top 10F′ bacteria. Multiple products were obtained, with sizes ranging from 1.6 to 0.35 kb. In contrast, a single product was obtained from genomic DNA amplified by PCR with primers SL38 and SL43. Multiple PCR products were also generated with template cDNA obtained from reverse transcription of a full length RNA transcribed in vitro from cDNA clone MAGE-C1 (SEQ ID NO: 1). These results suggest reverse transcription artifacts are responsible for the discrepancy between genomic and cDNA clones, and that the natural mRNA species transcribed from the MAGE-C1 gene in melanoma line LB373-MEL must comprise the entire repetitive region as found in cosmid C7.2 as described supra. The sequence of a full-length cDNA of this natural mRNA is presented as SEQ ID NO: 9.

The repetitive region corresponds to a total of 18 direct repeats of 14 amino-acids (aa), 17 repeats of 21-aa, and 16 repeats of 16 aa. Gene MAGE-C1 shares maximum overall homology with gene MAGE-A10. However, comparison and alignment are made in FIGS. 2 and 3 with MAGE-A1, the most well-characterized gene of the MAGE family. Exon 1 of gene MAGE-C1 has no homologous counterparts in other MAGEs, but it is noteworthy that one Sp1 and two Ets consensus binding sites immediately precede the first exon, as has been described in MAGE-1 (De Smet et al., Immunogenetics 42:282-290, (1995); De Smet et al., Proc. Natl. Acad. Sci. USA 93:7149-7153, (1996)) and some MAGE-4 promoters (De Plaen submitted).

### EXAMPLE 9

### Chromosomal Localization of the MAGE-C1 Gene

Fluorescence in situ hybridization (FISH) experiments with cosmid C7.2 as a probe show that gene MAGE-C1 is located on the long arm of the X chromosome, on Xq27 band.

A human genomic cosmid probe for MAGE-C1 was used for fluorescence in situ hybridization. The entire MAGE-C1 cosmid clone was nick translated using Biotin-14 dATP and Biotin-14 dCTP (Gibco BRL) for fluorescence in situ hybridization and hybridized to normal human metaphase spreads in two independent experiments.

Chromosome preparations were obtained from phytohemagglutinin-stimulated normal peripheral blood lymphocytes cultured for 72 hours. To induce R-banding, some of the cultures were synchronized with thymidine after 48 hours, incubated at 37° C. and treated with 5′bromodeoxyuridine (BrdU) the next morning, during the final late S-phase, and harvested 6 hours later (Jacky, P. B., Raven Press, p. 89, (1991)). Cytogenetic harvests and slide preparations were performed using standard methods. The slides were stored at −80° C. before use.

Fluorescence in situ hybridization to metaphase chromosomes was performed as described by Pinkel et al. (Pinkel et al., Proc. Natl. Acad. Sci. USA 83:2934-2938, (1986) incorporated herein by reference). Briefly the biotin-labeled probe (50-100 ng) was dissolved in hybridization mixture (50% formamide, 10% dextran sulfate, 2× SSC, 0.1 μg COT-1 DNA (Gibco BRL), 10 μg sheared salmon sperm DNA as carrier) and incubated for 60 min. at 37° C. to allow the COT-1 DNA to anneal to repetitive sequences in the probe. The probe mixture was then applied to the slide and co-denatured for 10 minutes at 80° C. on a slide warmer. Hybridization was allowed to proceed overnight in a humid chamber at 37° C. The slides were washed using the formamide-wash procedure as per the FITC-biotin detection kit and, when appropriate, the amplification protocol for dual color FISH (Oncor). Biotin-labeled probe detection was accomplished by incubation with the FITC-avidin conjugate and the digoxigenin-labeled chromosome X specific α-satellite repeat probe was detected using an anti-digoxigenin-rhodamine conjugate.

Chromosome identification was performed by simultaneous hybridization with a chromosome X-specific α-satellite repeat probe (Oncor) or by R-banding using 5-bromodeoxyuridine and mounting the slides in a modified antifade mounting solution of p-phenylenediamine (pH11) (Lemieux et al., Cytogenet. Cell Genet. 59:311-312 (1992)) containing 0.01 μg/ml propidium iodide as counterstain to produce an R-banding pattern. Slides were examined and photographed using a Zeiss Axiophot microscope and appropriate UV-filter combinations. The 35 mm slides were scanned using a Nikon Coolscan, processed using Adobe Photoshop 4.0 and printed using a Fujix Pictrography 3000.

The chromosomal localization of the human MAGE-C1 locus was initially obtained by somatic cell hybrid mapping in experiments not described here and was independently confirmed and refined by fluorescence in situ hybridization as described, supra. In these experiments, 47 R-banded metaphase spreads from normal lymphocytes were examined for specific signals of hybridization. Signals were considered to be specific only if they were detected on each chromatid of a single chromosome. Specific signals were seen in 15 of the 47 metaphases examined (32%). In each case the hybridization signals were located in the distal portion of the X chromosome. The R-banding pattern chromosomes allowed a more specific localization of the MAGE-C1 locus to Xq26-q27.

Interestingly, other members of the MAGE family have also been localized to both the long and short arms of the X chromosome. Twelve MAGE family genes have been mapped to the distal region of the long arm of the X chromosome (De Plaen, et al., Immunogenetics 40:360-369, (1994); Oaks et al., Cancer Research 54:1627-1629, (1994)) and MAGE-Xp is located in the Xp21.3 region of the short arm in the region (Muscatelli et al., Proc. Natl. Acad. Sci. USA 92:4987-4991(1995)).

### EXAMPLE 10

### Identification of Potential HLA Class I-Binding MAGE-C1 Peptides

Searching the MAGE-C1 protein sequence for HLA class I-binding peptides was performed on the Web site: http://bimas.dcrt.nih.gov/molbio (Parker, K. C., M. A. Bednarek, and J. E. Coligan, “Scheme for Ranking Potential HLA-A2 Binding Peptides Based on Independent Binding of Individual Peptide Side-Chains,” J. Immunol. 152:163 (1994) incorporated in its entirety by reference). Table 3 lists peptides expected to bind to the indicated HLA class I molecules and found more than once in the MAGE-C1 protein.

### EXAMPLE 11

A. Generation of Difference Products From Melanoma Tumor LB373-MEL.

A cDNA library enriched for sequences present only in melanoma cells, named tester, was generated by removing sequences that are shared with normal skin cells, called driver. The enrichment result is called a difference product (DP).

More precisely, total RNA was prepared from melanoma LB373-MEL cells (tester cells) and from a normal skin sample (driver cells), then purified on oligo-dT columns to obtain poly-A+ RNA. Poly-A+ RNA was reverse transcribed to produce cDNA. The resulting cDNA was digested with restriction enzyme DpnII, which cuts DNA at GATC sites, generating short fragments of double stranded DNA with 5′-GATC overhangs. Double stranded adaptors with a 5′-GATC overhang (R-Bgl adaptor which is composed of annealed R-Bgl 12 and R-Bgl 24, SEQ ID NOS: 2 and 4 respectively) were ligated to the digested cDNA. The adaptor-ligated cDNA was subsequently amplified by PCR, using as primer one strand of the R-Bgl adaptor, R-Bgl 24. The resulting product is called a representation of the tester and the driver, respectively. Both representations were digested with DpnII. Digested tester was ligated to new adaptor molecules (J-Bgl adaptor which is composed of annealed J-Bgl 12 and J-Bgl 24, SEQ ID NOS: 3 and 12 respectively). A first round of subtractive hybridization was then performed by mixing the digested driver cDNA with this J-Bgl adapted tester cDNA in 100/1 proportions, denaturing the sample and incubating at 67° for 20 hours to rehybridize the denatures sample. After hybridization, the sample was PCR amplified using one strand of the J-Bgl adaptor as primer, J-Bgl 24. Hybrids constituted by two DNA strands originating from the driver population could not be amplified, as they are not ligated to the J-Bgl adaptor, while hybrids constituted by one DNA strand of each origin (tester and driver) could only be amplified linearly. Only double strands with two tester strands (representing sequences unique to the tester) were amplified exponentially. After 10 cycles of PCR amplification, the sample was treated with Mung Bean Nuclease (which digests specifically the single stranded DNA produced by the linear amplification), then submitted to 18 additional PCR cycles.

The resulting product was called difference product 1 (DP1). J-Bgl adapters on DP1 were changed for N-Bgl-12/24 adapters (N-Bgl-12: 5′GATCTTCCCTCG-3′; N-Bgl-24: 5′-AGGCAACTGTGCTATCCGAGGGAA-3′), i.e., annealed N-Bgl-12 and N-Bgl-24 oligonucleotides, SEQ ID NO: 4 and SEQ ID NO: 13 and the process of subtractive hybridization and selective amplification repeated to generate the second difference products (except that annealing and extension in PCR reactions were performed at 72° C.). Tester to driver ratio was 1:800 to generate DP2.LB373(-skin).

DP2.LB373[-skin] was cloned in phagemid vector pTZ18R, to generate a cDNA library enriched in sequences expressed in melanoma but silent in normal skin.

B. Analysis of the Melanoma Enriched Library by Sequencing of Individual Clones.

49 individual clones isolated from the enriched melanoma library were sequenced. They correspond to 27 different genes. Search for homologies with sequences reported in databanks showed that 16 out of these 27 genes correspond to previously identified genes. Notably, two of them corresponded to gene MAGE-A3 and gene MAGE-A10, respectively, which are known to be expressed exclusively in tumors and in testis. Eleven sequences were unknown, and RT-PCR was used to determine whether they were expressed in a panel of different normal tissues. Only two out of these eleven new genes were not expressed in normal tissues except testis. The first one was named LAGE-1 and is described in U.S. patent application Ser. No. 08/791,495. The second one shares significant homologies with members of the MAGE gene family, and more particularly with gene MAGE-C1. It was therefore named MAGE-C2.

C. Search For a Complete MAGE-C2 cDNA.

The MAGE-C2 clone isolated from the enriched melanoma library is a DpnII restriction fragment of the complete MAGE-C2 messenger. To isolate a complete MAGE-C2 cDNA, we screened a cDNA library with a MAGE-C2 probe.

The cDNA library was constructed with LB373-MEL RNA in pcDNA1/Amp as described supra. Approximately 84,000 bacteria were plated on nylon membranes.

Duplicates were made and treated to denature and fix the bacterial DNA. A MAGE-C2 specific probe was generated by performing PCR on the partial MAGE-C2 clone with specific primers SL102 and SL103 (SL102: 5′AGGCGCGAATCAAGTTAG-3′, SEQ ID NO: 15; SL103: 5′CTCCTCTGCTGTGCTGAC-3′, SEQ ID NO: 16). The 206 bp MAGE-C2 PCR product was purified on a sepharose CL-6B column, then labeled using random primers, Klenow DNA polymerase and α-³²P-dCTP. Treated duplicates were hybridized with the MAGE-C2 specific probe (500,000 cpm/ml; overnight incubation at 65°), then washed in stringent conditions (last wash performed at 65° C. in SSC 0.2×, SDS 0.1%), and autoradiographed for 70 hours. Eight positive spots resulted. A secondary screening was carried out, and a bacterial clone was obtained which contained a large open reading frame for MAGE-C2.

The MAGE-C2 cDNA is 1983 bp-long (SEQ ID NO: 18). The open reading frame starts with an ATG at position 330, and ends with a stop codon at position 1449, coding for a putative protein of 373 amino-acids (SEQ ID NO: 19).

D. Structure of the MAGE-C2 Gene.

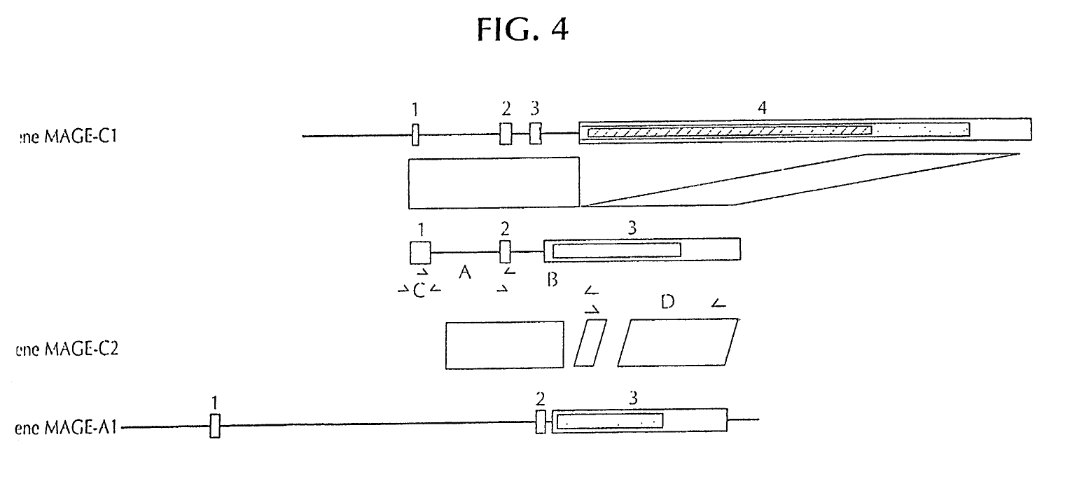

PCR primers complementary to several regions of the MAGE-C2 cDNA were selected and the PCR products obtained after amplification of cDNA and of genomic DNA were analyzed by agarose gel electrophoresis. PCR amplification of genomic DNA with primer pair A (SL 102:SEQ ID NO: 15 and SL 103:SEQ ID NO: 16) and primer pair B (SL122 5′-GGCTCCAGGAACCAGGT-3′ SEQ ID NO: 27 and SL 124 5′-TGCTC TCGGTAAGATTTGGT-3′ SEQ ID NO: 28) (FIG. 4) yielded products larger in size than those obtained by amplification of cDNA, revealing the existence of at least two introns in the MAGE-C2 gene. The sequences of these two introns were determined by sequencing of the PCR products. PCR amplification with primer pair C (SL125 5′-GGAATCTGACGGATCGGA-3′ SEQ ID NO: 29 and SL126 5′-CAGTCTCACGGCAGCGCA-3′:SEQ ID NO: 30) and primer pair D (SL114 5′-ACCAAATCTTACCGAGAGCA-3′ SEQ ID NO: 31 and SL115 5′-TGGCATCAT CTGCGGTATCA-3′ SEQ ID NO: 32) (FIG. 4) yielded products of identical sizes when cDNA or genomic DNA were used as templates, suggesting that no additional intron existed in the MAGE-C2 gene. The sequence of gene MAGE-C2, as deduced from the sequence of the cDNA clone and from the sequences of the introns, is shown in SEQ ID NO: 20.

Schematic representations of genes MAGE-C2, MAGE-C1 and MAGE-A1 are shown on FIG. 4. The entire MAGE-C2 gene is homologous to gene MAGE-C1 sequences. Nonetheless, MAGE-C2 does not contain the large repetitive region that is found in the coding region of gene MAGE-C1. The exon-intron structure of gene MAGE-C2 is intermediate between that of gene MAGE-C1 and that of MAGE-A genes, represented on FIG. 4 by gene MAGE-A1. Like the MAGE-A genes, MAGE-C2 comprises three exons, but MAGE-C2 exons 1 and 2 are homologous to MAGE-C1 exons 1 and 2. The third exon of MAGE-C2 has a structure comparable to that of the third exon of the MAGE-A genes: It contains the entire open reading frame, and it starts at a similar location. The coding sequence of gene MAGE-C2 is longer than that of gene MAGE-A 1, and this is due to the insertion, shortly after the start codon, of a 108 bp-sequence not found in gene MAGE-A1.

E. Expression of Gene MAGE-C2.

The expression pattern of the MAGE-C2 gene was determined by RT-PCR analysis of normal tissue and tumor samples. Selected sense primer SL102 (SEQ ID NO: 15) and antisense primer SL103 (SEQ ID NO: 16) are located in different exons (FIG. 4: primer pair A). MAGE-C2 is not expressed in a panel of normal tissues tested (Table 4), with the exception of testis. Among tumoral samples, MAGE-C2 is frequently expressed in melanoma and bladder transitional-cell carcinoma. It is also expressed in a significant fraction of head and neck carcinoma, breast carcinoma, non-small cell lung carcinoma, and sarcoma (Table 5). MAGE-C2 expression is correlated with that of other MAGE genes. 326 tumor samples were tested. Among the 63 samples that express gene MAGE-C2, 62 express also at least one MAGE-A gene. The only tumor sample that is positive for MAGE-C2 expression but negative for all other MAGE genes is a breast tumor sample. For cancer patients bearing tumors such as the latter, specific immunotherapy with MAGE antigens will rely solely on the use of MAGE-C2 derived antigens.

F. Chromosomal Location of the MAGE-C2 Gene.

The chromosomal location of the MAGE-C2 gene was determined by PCR analysis of the GeneBridge 4 Radiation Hybrid Panel (Walter et al., Nature Genet, 7:22-28 (1994) incorporated in its entirety by reference). Each DNA from the panel was submitted to PCR with primers SL102 and SL103 SEQ ID NOS: 15 and 16. PCR products were separated by agarose gel electrophoresis, blotted on a nitrocellulose membrane, and hybridized with radiolabeled primer SL118 (5′-AGCTGCCTCTGGTTGGCAGA-3′ SEQ ID NO: 17). Primer SL118 is complementary to a sequence of the first intron of gene MAGE-C2. PCR results were submitted to analysis on the web site, http://www-genome.wi.mit.edu/cgi-bin/contig/rhmapper.pl. The analysis revealed that MAGE-C2 is located on the X chromosome, between markers DXS 1227 and DSX7087. Gene MAGE-C1 is located between those same markers, which correspond to cytogenetic bands Xq26-Xq27.

G. MAGE-C2 and Other MAGE Proteins.

The MAGE-C2 protein shares similarities with other MAGE proteins. Multiple alignments of all known MAGE proteins show that maximal homology is observed on their COOH-terminus. Results of pairwise comparisons between the C-terminal two thirds of MAGE-C2 and the corresponding segments of other MAGE proteins are shown in Table 6. C-terminal segments of MAGE-A proteins share 52 to 94% amino-acid identity, and are closer in identity to each other than they are to MAGE-B proteins, with which they share 39 to 55% amino-acid identity. Similarly, MAGE-B proteins with 52 to 67% amino-acid identity, are closer to each other than they are to MAGE-A proteins. Based on a criteria of sequence similarity, MAGE-C1 and MAGE-C2 belong to a third subfamily: they share 68% amino-acid identity with each other, while sharing only 43 to 55% amino-acid identity with MAGE-A proteins and 39 to 46% with MAGE-B proteins. FIG. 2 demonstrates the MAGE-C2 gene is homologous to the MAGE-C1 gene (73-77% nt sequence identity.)

H. Identification of Potential HLA Class I-Binding MAGE-C2 Peptides.

Searching the MAGE-C2 protein sequence for HLA class I-binding peptides was performed on the Web site: http://bimas.dcrt.nih.gov/molbio. Table 7 lists MAGE-C2 peptides expected to bind to the indicated HLA class I molecules. These HLA class I molecules were shown previously on some tumors to present peptides encoded by a gene of the MAGE family.

I. Southern Blot Analysis.

A Southern blot prepared with genomic DNAs from melanoma cell lines LB373-MEL, SK29-MEL and LB33.A-1 was hybridized with a 1.9 kb PCR amplified probe derived from SEQ ID NO: 18. Preparation of the blot was performed as described supra (Example 7). Hybridization to the [α-³²P]dCTP radiolabeled MAGE-C2 probe was performed in 5× SSC, 5× Denhardt's, 0,1% SDS and 100 μg/ml denatured salmon sperm DNA for 18 hours at 68° C. Membranes were washed consecutively in 2× SSC, 0,1% SDS for 20 min. at room temperature, and in 2× SSC, 0,1% SDS for 20 min. at 68° C. Autoradiography was performed for 10 days.

Several hybridizing bands were found in the genomic DNAs obtained from the three melanoma lines. The genomic DNA were digested with BamHI or EcoRI restriction enzymes. In genomic DNAs digested with EcoRI, at least 5 bands hybridizing with the MAGE-C2 probe can be distinguished. Two of these were found to represent fragments of genes MAGE-C1 and MAGE-C2, respectively. These results suggest that MAGE-C1 and MAGE-C2, described herein, are members of a larger MAGE-C family.

### EXAMPLE 12 MAGE-C3

Analysis of the genomic nucleotide sequence data that was produced by the Human Chromosome X Sequencing Group at the Sanger Center, and can be obtained from http://www.ncbi.nlm.nih.gov/Entrez/nucleotide.html/ under accession number AL022152, led to the identification of MAGE-C3. The sequence under accession number AL022152 is from clone 232G24 of the P1 artificial chromosome library RPCI6 constructed at the Roswell Park Cancer Institute by the group of Pieter de Jong (for further details see http://bacpac.med.buffalo.edu/). This PAC clone has been mapped to Xq27.1-Xq27.3, a region of the X chromosome that also contains genes MAGE-C1 and MAGE-C2. An open reading frame (ORF) that encompasses nucleotides 3761 to 4801 of the PAC clone encodes a protein of 346 amino-acids that shares similarities with all known MAGE proteins. Amino acid sequence comparison was performed using MacVector™ 6.5(Oxford Molecular Ltd., Oxford, England) and ClustalW (J. D. Thompson et al., “Improving the sensitivity of progressive multiple sequence alignment through sequence weighting, positions-specific gap penalties and weight matrix choice”, Nucl. Acids. Res., 22:4673-4680 (1994)). The program parameters were as follows: matrix=Biosum 30, alignment speed=slow, and open gap and extend gap penalties were 10.0 and 0.1 respectively. A maximum homology, i.e., identity, of 56% was observed between this 346 amino acid protein and proteins MAGE-C1 and MAGE-C2. Therefore, the gene encoding this protein was named MAGE-C3. FIG. 5 depicts the ORF of the MAGE-C3 gene. FIG. 6 depicts the amino acid sequence of the encoded MAGE-C3 protein.

We designed an RT-PCR assay to analyze the pattern of expression of MAGE-C3 using sense primer SL 164 and anti-sense primer SL 165. Selected sense primer SL 164 (5′-TCATCCTCACCCTTGTCCTCA-3′, nt 175-195 SEQ ID NO: 21) and antisense primer SL165 (5′-CCCTGGTCATCAATCAGGCTT-3′, nt 711-731 SEQ ID NO: 21) are shown in FIG. 5. PCR was conducted essentially as described by P. Weynants et al., “Expression of MAGE genes by non-small-cell lung carcinomas”, Int. J. Cancer, 56:826-829 (1994) incorporated herein by reference. Briefly, cDNA produced from 50 ηg of total RNA or 25-100 ηg of genomic DNA from the sample to be assayed was PCR amplified in a TRIO™-Thermoblock (Biometra, Göttingen, Germany) for 30 cycles of 1 min. at 94° C.; 1 min. at 65° C., and; 3 min at 72° C. PCR products were analyzed by agarose gel electrophoresis. The PCR products obtained by amplification of testis cDNA were sequenced to confirm the specificity of the primers.

PCR products obtained on genomic DNA of LB23-SARC sarcoma cell line and testis cDNA are of identical sizes (556 bp), which demonstrates that the primers are located in a single exon. Because our assay cannot distinguish amplification of cDNA from amplification of occasionally contaminating genomic DNA, all RNA samples that were positive in the MAGE-C3 RT-PCR assay, were controlled for contaminating DNA by PCR amplification in the absence of reverse transcription. No amplification was detected indicating that the samples were not contaminated with DNA. MAGE-C3 is not expressed in a panel of normal tissues tested (Table 8), with the exception of testis. Among tumoral samples (Table 9), MAGE-C3 is expressed in a significant fraction of seminoma, bladder transitional-cell carcinoma and sarcoma. It is also expressed in some melanoma and non small-cell lung carcinoma samples.

The cDNA nucleotide sequences of MAGE-C3 (SEQ ID NO: 21) with the MAGE-C1 (SEQ ID NO: 9) and MAGE-C2 (SEQ ID NO: 18) were compared using tblastn (version BLASTN 2.0.9: match (1), mismatch (−2), gap open (5), gap extension (2), dropoff (50), expect (10.0) wordsize (11) with filter). An identity of 83% between MAGE-C3 and MAGE-C1 and an identity of 79% between MAGE-C3 and MAGE-C2 was found. About 80% identity was found between nt 472-1024 of SEQ ID NO: 21 and MAGE-C1 and MAGE-C2.

### EXAMPLE 13

### MAGE-BS and MAGE-B6, Two New Members of the MAGE-B Family

Two other MAGE-type genes were identified in genomic nucleotide sequence data that was produced by the Human Genome Sequencing Center at the Baylor College of Medicine (Houston, Tex., USA) and can be obtained under accession number AC005297. The sequence is from clone 46604 of the Bacterial Artificial Chromosome library RPCI11 constructed at the Roswell Park Cancer Institute by the group of Pieter de Jong. This BAC clone has been mapped to Xp22, a region of the X chromosome located near the region that contains the four known genes of the MAGE-B family. Two open reading frames located in the sequence of the BAC clone encode proteins that share similarities with other known MAGE proteins. Maximum homology is observed with proteins of the MAGE-B family, and therefore, the two genes were named MAGE-B5 and MAGE-B6.

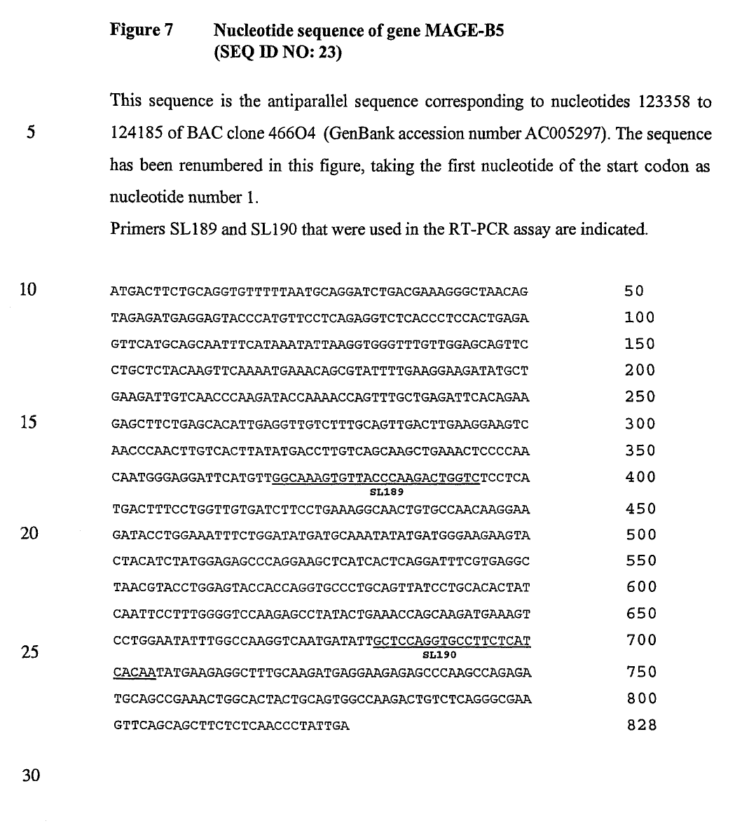

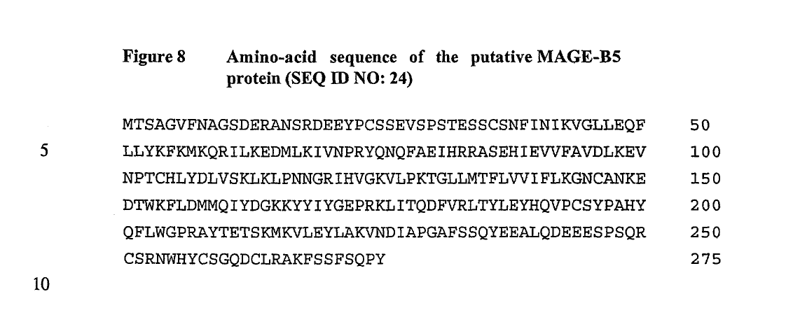

The open reading frame (ORF) of gene MAGE-B5 begins with a start codon at nucleotide 124185 of the BAC clone, and ends with a stop codon at nucleotide 123358 of the BAC clone. FIG. 7 shows the nucleotide sequence of the ORF for MAGE-B5 and FIG. 8 shows the amino-acid sequence of the encoded MAGE-B5 protein. Sequence homology with other proteins was determined using MacVector 6.5 and ClustalW as described in Example 12. MAGE-B5 protein shares between 44 and 52% amino-acid identities with MAGE-B 1 to -B4 proteins respectively.

We designed an RT-PCR assay to analyze the pattern of expression of MAGE-B5. Selected sense primer SL189, nt 370-394 of SEQ ID NO: 23, and antisense primer SL190, nt 682-705 of SEQ ID NO: 23, are shown in FIG. 7. PCR was conducted essentially as described supra. cDNA produced from 50 ηg of total RNA or 25-100 ηg of genomic DNA was amplified with SL189 and SL190 in a TRIO™-Thermoblock (Biometra, Göttingen, Gemany) for 30 cycles of 1 min. at 94° C.; 1 min. at 68° C. and 3 min at 72° C. PCR products were analyzed by agarose gel electrophoresis. A PCR product of the expected size (334 bp) was obtained by amplification of genomic DNA from LB23-SARC sarcoma cell line, showing that our primers are able to amplify specifically MAGE-B5. MAGE-B5 is not expressed in a panel of normal tissues tested (Table 10), including testis. Among 64 tumoral samples tested, MAGE-B5 expression was observed in one seminoma sample only (Table 11).

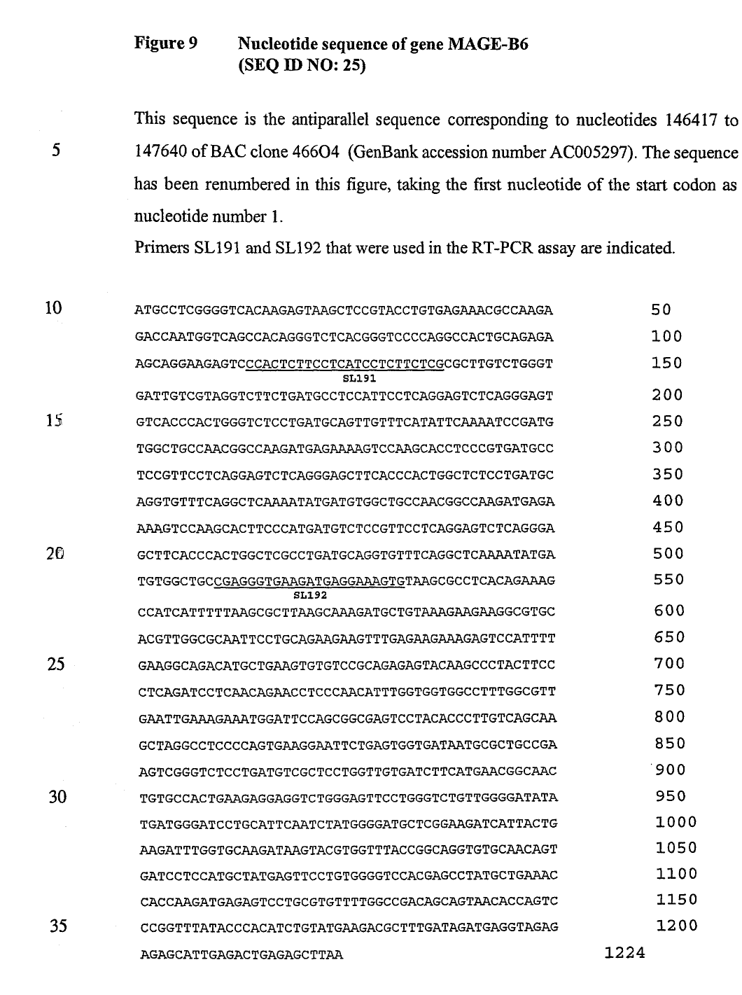

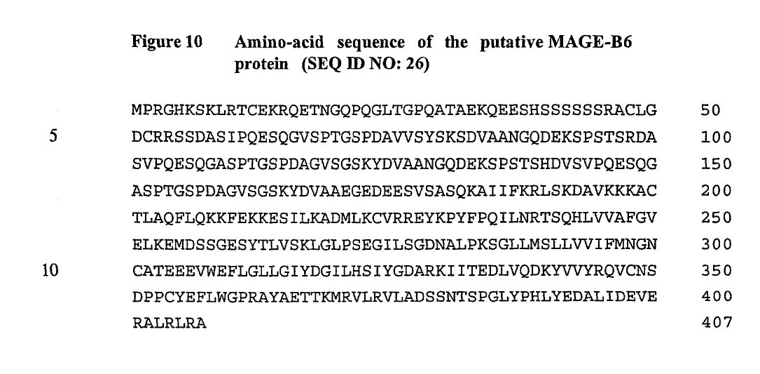

The open reading frame (ORF) of MAGE-B6 (SEQ ID NO: 25) begins with a start codon at nucleotide 147640 of the BAC clone, and ends with a stop codon at nucleotide 146417 of the BAC clone. FIG. 9 depicts the ORF of the MAGE-B6 gene and FIG. 10 shows the amino-acid sequence of the encoded MAGE-B6 protein (SEQ ID NO: 26). The MAGE-B6 protein shares between 41 and 46% amino-acid identities with MAGE-B1 to MAGE-B4 proteins, respectively as determined using MacVector 6.5 and ClustalW as described supra.

We designed an RT-PCR assay to analyze the pattern of expression MAGE-B6. Selected sense primer SL191 (nt 114-137 SEQ ID NO: 25) and antisense primer SL192 (nt 510-532 SEQ ID NO: 25) are shown in FIG. 9. PCR was conducted essentially as described supra. cDNA produced from 50 ηg of total RNA or 25-100 ηg of genomic DNA from the sample to be assayed was amplified with SL191 and SL192 in a TRIO™-Thermoblock (Biometra, Göttingen, Germany) for 30 cycles of 1 min. at 94° C.; 1 min. at 68° C. and 3 min at 72° C. PCR products were analyzed by agarose gel electrophoresis. The PCR products obtained by amplification of testis cDNA were sequenced to confirm the specificity of the primers. PCR products from the genomic DNA sample and the testis cDNA sample were of identical sizes (418 bp), which means that the primers are located in a single exon. Because our assay cannot distinguish amplification of cDNA from amplification of occasionally contaminating genomic DNA, all RNA samples that were positive in the MAGE-B6 RT-PCR assay, were controlled for contaminating DNA by PCR amplification in the absence of reverse transcription. MAGE-B6 is not expressed in a panel of normal tissues tested (Table 12), with the exception of testis. Among tumoral samples (Table 13), MAGE-B6 is expressed in a significant fraction of seminoma. It is also expressed in some samples from melanoma, esophageal carcinoma and non small-cell lung carcinoma.

Three direct tandem repeats of 126 nucleotides were found in the open reading frame of gene MAGE-B6, starting 162 nucleotides after the start codon. Variation in repeat numbers in gene MAGE-C1 can cause allelic polymorphism (Example 7 and Lucas et al., Cancer Res., 58, 643-752 (1998). Variation in the repeat number were also detected in MAGE-B6 from two different patients. Genomic DNA from two different patients was PCR amplified with primers SL191 and SL192, which are located on either side of the repetitive region, and the PCR products from the patients were sequenced. Three repeats were found in the MAGE-B6 sequences of one patient, exactly as was observed in the genomic BAC clone, while only two repeats were found in the second patient. Thus allelic polymorphism is produced by variation in repeat number in both MAGE-C1 and MAGE-B6.

TABLE 1
MAGE-C1 Expression Determined By RT-PCR On
Normal Tissue Samples.

| | number of samples |
|---|---|
| | expressing MAGE-C1/ |
| Type of tissue | number of samples assayed |
| Bladder | 0/2 |
| Brain | 0/4 |
| Breast | 0/3 |
| Colon | 0/2 |
| Epididymis | 0/1 |
| Kidney | 0/1 |
| Liver | 0/4 |
| Lung | 0/6 |
| Lymphocytes (PBL) | 0/4 |
| Ovary | 0/1 |
| Placenta | 0/1 |
| Prostate | 0/2 |
| Testis | 3/3 |
| Uterus | 0/4 |

TABLE 2
MAGE-C1 Expression Determined by RT-PCR On
Tumor Samples.

| | | Percent |
|---|---|---|
| | number of | expressing |
| | samples expressing MAGE-C1/ | MAGE- |
| Tumor type | number of samples assayed | C1 |
| Cutaneous melanoma | 48/105 | 46% |
| Primary | 17/46 | 37% |
| Metastatic | 31/59 | 52% |
| Mucosis melanoma | 5/8 | |
| Uveal melanoma | 0/9 | |
| Testicular tumors | | |
| Seminoma | 9/9 | 100% |
| Non-seminoma | 0/3 | |
| Neuroblastoma | 1/3 | |
| Bladder transitional-cell | 9/51 | 18% |
| carcinoma | | |
| Invasive | 9/37 | 24% |
| Superficial | 0/14 | |
| Breast carcinoma | 6/36 | 16% |
| Lung carcinoma | | |
| NSCLC | 15/95 | 16% |
| SCLC | 0/3 | |
| Sarcoma | 2/17 | 12% |
| Brain tumors | 1/9 | |
| Prostate adenocarcinoma | 2/18 | 11% |
| Head-and-neck squamous- | 4/42 | 10% |
| cell carcinoma | | |
| Colorectal carcinoma | 0/30 | |
| Leukemia | 0/37 | |
| Myeloma | 0/1 | |
| Renal tumors | 0/8 | |
| Pancreatic tumors | 0/1 | |
| Ovarian tumors | 0/3 | |
| Uterine tumors | 0/9 | |
| Esophageal carcinoma | 0/6 | |
| Mesothelioma | 0/3 | |

TABLE 3
Repeated Peptides Found in Protein MAGE-C1 and
Expected to Bind to HLA Class I Molecules, as
Determined By Analysis on Web Site
http://bimas.dcrt.nih.gov/molbio

| HLA Class I | | Start position in the | # of |
|---|---|---|---|
| molecule | MAGE-C1 peptide | MAGE-C1 protein | repetitions |
| B 60 | FEGFPQSPL | 190, 260, 365, 400, 435, | 7 |
| | | 470, 506 | |
| B 62 | LQIPVSRSF | 198, 268 | 2 |
| B 2705 | LQIPMTSSF | 338, 408 | 2 |
| | ERTQSTFEGF | 254, 289, 324, 464 | 4 |
| B 4403 | GEDSLSPHY | 556, 571, 586 | 3 |
| B 5101 or | FPSSTSSSL | 817,834 | 2 |
| B5102 | SPPQGEDSL | 551, 567 | 2 |
| | EGFPQSPLQI | 191, 261, 366, 401,436, | 7 |
| | | 471, 507 | |
| | FPQSPLQIPV | 193, 263, 438, 473 | 4 |
| | EGFAQSPLQI | 226, 296 | 2 |
| | FAQSPLQIPV | 228, 298 | 2 |
| B 5103 | FAQSPLQIPV | 228,298 | 2 |
| B 5801 | RTQSTFEGF | 255, 290, 325, 265 | 4 |
| Cw 0401 | FPSSTSSSL | 817,834 | 2 |
| | TFEGFPQSPL | 259, 364, 399, 469, 505 | 5 |
| | SFSSTLLSIF | 205, 275, 345 | 3 |
| | SFPSSTSSSL | 833, 816 | 2 |

TABLE 4
MAGE-C2 Expression in Normal Tissues, As Analyzed By
RT-PCR With Primers SL102 and SL103.

| | MAGE-C2 |
|---|---|
| Type of Tissue | Expression |
| Bladder | − |
| Brain | − |
| Breast | − |
| Colon | − |
| Heart | − |
| Kidney | − |
| Liver | − |
| Lung | − |
| Lymphocytes (PBL) | − |
| Ovary | − |
| Placenta | − |
| Skin | − |
| Stomach | − |
| Suprarenals | − |
| Testis | + |
| Uterus | − |

TABLE 5
MAGE-C2 Expression in Tumoral Samples, As Analyzed
by RT-PCR With Primers SL102 and SL103

| | Number of Positive | |
|---|---|---|
| | Samples/Number | |
| Tumor Type | Tested | |
| Cutaneous Melanoma | 30/70 | (43%) |
| Primary | 10/30 | (33%) |
| Metastatic | 20/40 | (50%) |
| Uveal Melanoma | 0/5 | |
| Bladder Transitional-Cell Carcinoma | 9/30 | |
| Invasive | 6/15 | |
| Superficial | 3/15 | |
| Head-and-Neck Squamous-Cell | 4/20 | |
| Carcinoma | | |
| Breast Carcinoma | 3/20 | |
| Lung Carcinoma (NSCLC) | 4/35 | |
| Sarcoma | 2/15 | |
| Esophageal Carcinoma | 2/15 | |
| Prostate Adenocarcinoma | 1/10 | |
| Myeloma | 1/5 | |
| Brain Tumors | 0/9 | |
| Colorectal Carcinoma | 0/20 | |
| Leukemia | 0/25 | |
| Neuroblastoma | 0/2 | |
| Mesothelioma | 0/4 | |
| Renal Tumors | 0/24 | |
| Thyroid Tumors | 0/5 | |
| Uterine Tumors | 0/5 | |

TABLE 6
Percentage of Amino-Acid Identity Between C-Terminal Fragments of All Known
MAGE Proteins

| | A1 | A2 | A3 | A4 | A6 | A8 | A9 | A10 | A11 | A12 | B1 | B2 | B3 | B4 | C1 |
|---|---|---|---|---|---|---|---|---|---|---|---|---|---|---|---|
| A1 | | | | | | | | | | | | | | | |
| A2 | 68 | | | | | | | | | | | | | | |
| A3 | 68 | 83 | | | | | | | | | | | | | |
| A4 | 77 | 66 | 66 | | | | | | | | | | | | |
| A6 | 69 | 82 | 94 | 66 | | | | | | | | | | | |
| A8 | 73 | 64 | 83 | 77 | 63 | | | | | | | | | | |
| A9 | 65 | 58 | 60 | 68 | 57 | 72 | | | | | | | | | |
| A10 | 63 | 56 | 52 | 60 | 55 | 64 | 59 | | | | | | | | |
| A11 | 62 | 56 | 56 | 62 | 56 | 62 | 63 | 62 | | | | | | | |
| A12 | 67 | 86 | 83 | 65 | 81 | 65 | 58 | 54 | 56 | | | | | | |
| B1 | 45 | 40 | 39 | 46 | 39 | 43 | 43 | 47 | 46 | 41 | | | | | |
| B2 | 43 | 40 | 40 | 43 | 39 | 42 | 43 | 45 | 42 | 40 | 62 | | | | |
| B3 | 50 | 40 | 40 | 47 | 40 | 46 | 46 | 48 | 48 | 41 | 52 | 55 | | | |
| B4 | 50 | 44 | 43 | 47 | 44 | 48 | 49 | 55 | 50 | 45 | 67 | 64 | 59 | | |
| C1 | 49 | 44 | 44 | 49 | 46 | 50 | 50 | 53 | 49 | 44 | 39 | 44 | 40 | 43 | |
| C2 | 50 | 46 | 46 | 49 | 46 | 49 | 50 | 55 | 50 | 43 | 43 | 46 | 44 | 46 | 68 |

TABLE 7
Peptides Found in Protein MAGE-C2 and Expected to
Bind to the Indicated HLA Class I Molecules, as
Determined by Analysis on Web Site
http://bimas.dcrt.nih.gov/molbio

| | | Start Position in the |
|---|---|---|
| | | MAGE-C2 Protein (SEQ |
| HLA Class I Molecule | MAGE-C2 Peptide | ID NO: 19) |
| A1 | LVEFLLLKY | 148 |
| | YGEPRELLTK | 267 |
| A0201 | VIWEVLNAV | 248 |
| | KVLEFLAKL | 313 |
| | SLLIIILSV | 228 |
| | FLAKLNNTV | 317 |
| | KVWVQGHYL | 276 |
| | KVAELVEFL | 144 |
| | LLFGLALIEV | 191 |
| | GLPDSESSFT | 129 |
| | KVAELVEFLL | 144 |
| | GVYAGREHFV | 257 |
| B4403 | AEMLMIVIKY | 165 |
| | WEVLNAVGVY | 250 |
| | REVPHSSPPY | 287 |
| | DEKVAELVEF | 142 |

TABLE 8
MAGE-C3 expression in normal tissues, as analyzed by
RT-PCR with primers SL164 and SL165.

| Type | MAGE-C3 | Contaminating DNA |
|---|---|---|
| of Tissue | expression | in positive samples |
| Bladder | − | |
| Brain | − | |
| Breast | − | |
| Colon | − | |
| Heart | − | |
| Kidney | − | |
| Liver | − | |
| Lung | − | |
| Lymphocytes(PBL) | − | |
| Ovary | − | |
| Placenta | − | |
| Skin | − | |
| Stomach | − | |
| Suprarenals | − | |
| Testis | + | no |
| Uterus | − | |

TABLE 9
MAGE-C3 expression in tumoral samples, as analyzed by
RT-PCR with primers SL164 and SL165

| | number of positive | Contaminating DNA |
|---|---|---|
| Tumor type | samples/number tested | in positive samples |
| Sentinoma | 4/5 | no |
| Bladder transitional- | 2/8 | no |
| cell carcinoma | | |
| Sarcoma | 2/5 | no |
| Cutaneous melanoma | 1/10 | no |
| Lung carcinoma | 1/16 | no |
| (NSCLC) | | |
| Head-and-neck | 0/10 | |
| squamous-cell | | |
| carcinoma | | |
| Esophageal carcinoma | 0/10 | |

TABLE 10
MAGE-B5 expression in normal tissues, as analyzed by
RT-PCR with primers SL189 and SL190.

| Type | MAGE-B5 |
|---|---|
| of Tissue | expression |
| Bladder | − |
| Brain | − |
| Breast | − |
| Colon | − |
| Heart | − |
| Kidney | − |
| Liver | − |
| Lung | − |
| Lymphocytes(PBL) | − |
| Ovary | − |
| Placenta | − |
| Skin | − |
| Stomach | − |
| Suprarenals | − |
| **Testis | − |
| Uterus | − |

TABLE 11
MAGE-B5 expression in tumoral samples, as analyzed by
RT-PCR with primers SL189 and SL190.

| | number of positive | Contaminating DNA |
|---|---|---|
| Tumor type | samples/number tested | in positive samples |
| Seminoma | 1/5 | no |
| Bladder transitional- | 0/8 | |
| cell carcinoma | | |
| Sarcoma | 0/5 | |
| Cutaneous melanoma | 0/10 | |
| Lung carcinoma | 0/16 | |
| (NSCLC) | | |
| Head-and-neck | 0/10 | |
| squamous-cell | | |
| carcinoma | | |
| Esophageal carcinoma | 0/10 | |

TABLE 12
MAGE-B6 expression in normal tissues, as analyzed by
RT-PCR with primers SL191 and SL192.

| Type of | MAGE-B6 | Contaminating DNA |
|---|---|---|
| Tissue | expression | in positive samples |
| Bladder | − | |
| Brain | − | |
| Breast | − | |
| Colon | − | |
| Heart | − | |
| Kidney | − | |
| Lung | − | |
| Lymphocytes (PBL) | − | |
| Ovary | − | |
| Placenta | − | |
| Skin | − | |
| Stomach | − | |
| Adrenals | − | |
| Testis | + | no |
| Uterus | − | |

TABLE 13
MAGE-B6 expression in tumoral samples, as analyzed by RT-PCR
with primers SL191 and SL192

| | number of positive | Contaminating DNA |
|---|---|---|
| Tumor type | samples/number tested | in positive samples |
| Seminoma | 4/5 | no |
| Cutaneous melanoma | 1/10 | no |
| Lung carcinoma | 1/16 | no |
| (NSCLC) | | |
| Esophageal carcinoma | 1/10 | no |
| Head-and-neck | 0/10 | |
| squamous-cell carcinoma | | |
| Sarcoma | 0/5 | |
| Bladder transitional-cell | 0/8 | |
| carcinoma | | |

We claim:

1. An isolated nucleic acid molecule which encodes a tumor rejection antigen precursor “TRAP” having an amino acid sequence of a TRAP encoded by a nucleotide sequence selected from the group consisting of SEQ ID NO: 21, SEQ ID NO: 23 and SEQ ID NO: 25.

2. An isolated nucleic acid molecule having a nucleotide sequence which encodes a tumor rejection antigen precursor, said isolated nucleic acid molecule comprising a nucleotide sequence selected from the group consisting of SEQ ID NO: 21, SEQ ID NO: 23 and SEQ ID NO: 25.

3. The isolated nucleic acid molecule of claim 1, wherein said nucleic acid molecule is a cDNA molecule.

4. The isolated nucleic acid molecule of claim 1, wherein said nucleic acid molecule is a genomic DNA molecule.

5. The isolated nucleic acid molecule of claim 1, wherein said nucleic acid molecule is an isolated mRNA molecule.

6. An expression vector comprising the isolated nucleic acid molecule according to claim 1 operatively linked to a promoter.

7. An expression vector comprising the isolated nucleic acid molecule according to claim 3 operably linked to a promoter.

8. The expression vector according to claim 6, wherein the promoter is an inducible promoter.

9. A cell line or cell strain transfected or transformed with the expression vector of claim 6.

10. A cell line or cell strain transfected or transformed with the expression vector of claim 7.

11. The cell line according to claim 9, wherein said cell line is a eukaryotic cell line.

12. The cell line according to claim 11, wherein said cell line is selected from the group consisting of a human cell line, a rodent cell line and a simian cell line.

13. The cell line according to claim 12, wherein said cell line is selected from the group consisting of a COS cell line and a CHO cell line.

14. The cell line according to claim 10, wherein said cell line is a eukaryotic cell line.

15. The cell line according to claim 14, wherein said cell line is selected from the group consisting of a human cell line, a rodent cell line or a simian cell line.

16. The cell line according to claim 15, wherein said cell line is selected from the group consisting of a COS cell line and a CHO cell line.

17. A method for determining presence of cytolytic T cells specific for complexes of an HLA molecule and a peptide derived from the protein encoded by the isolated nucleic acid molecule of claim 1 in a CTL-containing sample, comprising contacting said sample with cells which present said complexes on their surface, and determining (i) proliferation of cytolytic T cells, or (ii) lysis of cells presenting said complexes as a determination of said cytolytic T cells in said sample.

18. The method according to claim 17, comprising determining proliferation of cytolytic T cells by measuring tumor necrosis factor release.

19. The method according to claim 17, comprising determining lysis of said cells by determining release of a radiolabelled substance from said cells.

20. The method according to claim 19, wherein the radiolabelled substance is ⁵¹Cr.

21. The method according to claim 17, wherein said cells which present said complexes have been transfected or transformed with at least one of (i) a nucleic acid molecule which codes for an HLA molecule and (ii) the isolated nucleic acid molecule of claim 1.

22. The method according to claim 17, wherein said cells have been transfected or transformed with both of(i) a nucleic acid molecule which codes for an HLA molecule and (ii) the nucleic acid molecule of claim 1.

23. An isolated tumor rejection antigen precursor encoded by the isolated nucleic acid molecule of claim 1.

24. An isolated tumor rejection antigen precursor encoded by the isolated nucleic acid molecule of claim 3.

25. A cell line or cell strain transfected or transformed with the isolated nucleic acid molecule of claim 1.

26. A polytope comprising a plurality of tumor rejection antigens wherein said antigens are derived from a tumor rejection antigen precursor selected from the group consisting of MAGE-C3, MAGE-B5, and MAGE-B6 tumor rejection antigen precursors.

27. The polytope of claim 26 comprising at least one other tumor rejection antigen derived from a TRAP selected from the group consisting of MAGE-C1 and MAGE-C2 TRAPs.

28. Kit useful in a polymerase chain reaction based assay, comprising an oligonucleotide having a sequence of nucleotides 175-195 of SEQ ID NO: 21 and an oligonucleotide having a nucleotide sequence that is complementary to nucleotides 711-731 of SEQ ID NO: 21.

29. Kit useful in a polymerase chain reaction based assay, comprising an oligonucleotide having a sequence of nucleotides 370-394 of SEQ ID NO: 23 and an oligonucleotide having a nucleotide sequence that is complementary to nucleotides 682-705 of SEQ ID NO: 23.

30. Kit useful in a polymerase chain reaction based assay, comprising an oligonucleotide having a sequence of nucleotides 114-137 of SEQ ID NO: 25 and an oligonucleotide having a nucleotide sequence that is complementary to nucleotides 510-532 of SEQ ID NO: 25.

31. Method for determining expression of a MAGE-C3 gene in a sample, comprising contacting said sample with (i) an oligonucleotide having a sequence set forth by nucleotides 175-195 of SEQ ID NO: 21 and (ii) an oligonucleotide having a sequence that is complementary to nucleotides 711-731 of SEQ ID NO: 21, under conditions favoring hybridization of the sequences of (i) or (ii) to an MAGE-C3 coding sequence, carrying out polymerase chain reaction and determining expression product to determine presence of an MAGE-C3 coding sequence in said sample.

32. Method for determining expression of a MAGE-B5 gene in a sample, comprising contacting said sample with (i) an oligonucleotide having a sequence set forth by nucleotides 370-394 of SEQ ID NO: 23 and (ii) an oligonucleotide having a sequence that is complementary to nucleotides 682-705 of SEQ ID NO: 23, under conditions favoring hybridization of the sequences of (i) or (ii) to an MAGE-B5 coding sequence, carrying out polymerase chain reaction and determining expression product to determine presence of an MAGE-B5 coding sequence in said sample.

33. Method for determining expression of a MAGE-B6 gene in a sample, comprising contacting said sample with (i) an oligonucleotide having a sequence set forth by nucleotides 114-137 of SEQ ID NO: 25 and (ii) an oligonucleotide having a sequence that is complementary to nucleotides 510-532 of SEQ ID NO: 25, under conditions favoring hybridization of the sequences of (i) or (ii) to an MAGE-B6 coding sequence, carrying out polymerase chain reaction and determining expression product to determine presence of an MAGE-B6 coding sequence in said sample.
